SEQUENCE LISTING (6 sequences)

SEQ ID NO: 1 - DNA, 50 bp, Artificial Sequence
  Synthetic Oligonucleotide
ctctcgtcaggttgaatggcatggtcgctggctggatgcagaaagctgga

SEQ ID NO: 2 - DNA, 57 bp, Artificial Sequence
  Synthetic Oligonucleotide
ccacaaagccattcccggcaaggttaggaacaacatcctgctgctttaatgctgcgg

SEQ ID NO: 3 - DNA, 50 bp, Artificial Sequence
  Synthetic Oligonucleotide
caccttcatggtggtcagtgcgtcctgctgatgtgctcagtatcaccgcc

SEQ ID NO: 4 - DNA, 51 bp, Artificial Sequence
  Synthetic Oligonucleotide
cctcagcgccgggttttctttgcctcacgatcgcccccaaaacacataacc

SEQ ID NO: 5 - DNA, 53 bp, Artificial Sequence
  Synthetic Oligonucleotide
gcggtgatgacgccgagccgtaatttgtgccacgcatcatccccctgttcgac

SEQ ID NO: 6 - DNA, 53 bp, Artificial Sequence
  Synthetic Oligonucleotide
ggattcctgaaacagaaagccgcagagcagaaggtggcagcatgacaccggac

(19) United States
(12) Patent Application Publication
(10) Pub. No.: US 2019/0360043 A1
(43) Pub. Date: Nov. 28, 2019

(54) ENRICHMENT OF DNA COMPRISING TARGET SEQUENCE OF INTEREST

(71) Applicant: Pacific Biosciences of California, Inc., Menlo Park, CA (US)

(72) Inventors: Thang Pham, Mountain View, CA (US); Stephen Turner, Eugene, OR (US); Keith Bjornson, Fremont, CA (US); Jeremiah Hanes, Woodside, CA (US)

(21) Appl. No.: 16/418,559

(22) Filed: May 21, 2019

Related U.S. Application Data
(60) Provisional application No. 62/675,352, filed on May 23, 2018.

(51) Int. Cl. C12Q 1/6874; C12Q 1/6855; C12Q 1/6876

(52) U.S. Cl. CPC C12Q 1/6874; C12Q 1/6855; C12Q 1/6876; C12Q 2521/301; C12Q 2521/319; C12Q 2563/185; C12Q 2525/191; C12Q 2525/131; C12Q 2525/155; C12Q 2525/161; C12Q 2563/179; C12Q 2525/301

(57) ABSTRACT

Disclosed are methods and compositions for enriching nucleic acid fragments from a sample that include one or more target region of interest. In certain aspects, a sample of double stranded nucleic acid fragments having a strand-linking adapter at one end and a non-strand-linking adapter at the other end are denatured and contacted with capture probes specific for a target sequence of interest. Capture probe-bound fragments are isolated from the sample, e.g., using a solid substrate specific for the binding moiety on the capture probes, and are renatured for downstream processing, thus maintaining the original double-stranded region. This enrichment process does not require amplification and as such maintains the nucleic acids in their native states. The disclosed enrichment process and compositions are suitable for analyzing nucleic acids that are fragmented and/or damaged, e.g., cell-free DNA such as circulating tumor DNA, as well as nucleic acids that are many kilobases in length.

### CROSS-REFERENCE TO RELATED APPLICATIONS

This application is a non-provisional utility patent application claiming priority to and benefit of provisional patent application U.S. Ser. No. 62/675,352, filed May 23, 2018, entitled “ENRICHMENT OF DNA COMPRISING TARGET SEQUENCE OF INTEREST” by Thang Pham et al., which is incorporated herein by reference in its entirety for all purposes.

### STATEMENT AS TO RIGHTS TO INVENTIONS MADE UNDER FEDERALLY SPONSORED RESEARCH AND DEVELOPMENT

Not applicable.

### INCORPORATION-BY-REFERENCE OF MATERIAL SUBMITTED BY U.S.P.T.O. EFS-WEB

The instant application contains a Sequence Listing which is being submitted in computer readable form via the United States Patent and Trademark Office eFS-WEB system and which is hereby incorporated by reference in its entirety for all purposes. The txt file submitted herewith contains a 2 KB file (01021701_2019-05-21_SequenceListing.txt).

### BACKGROUND OF THE INVENTION

The ability to read the genetic code has opened countless opportunities to benefit humankind. Whether it involves the improvement of food crops and livestock used for food, the identification of the causes of disease, the generation of targeted therapeutic methods and compositions, or simply the better understanding of what makes us who we are, a fundamental understanding of the blueprints of life is an integral and necessary component.

A variety of techniques and processes have been developed to obtain genetic information, including broad genetic profiling or identifying patterns of discrete markers in genetic codes and nucleotide level sequencing of entire genomes. With respect to determination of genetic sequences, while techniques have been developed to read, at the nucleotide level, a genetic sequence, such methods can be time-consuming and extremely costly.

Approaches have been developed to sequence genetic material with improved speed and reduced costs. Many of these methods rely upon the identification of nucleotides being incorporated by a polymerization enzyme during a template sequence-dependent nucleic acid synthesis reaction. In particular, by identifying nucleotides incorporated against a complementary template nucleic acid strand, one can identify the sequence of nucleotides in the template strand. A variety of such methods have been previously described. These methods include iterative processes where individual nucleotides are added one at a time, washed to remove free, unincorporated nucleotides, identified, and washed again to remove any terminator groups and labeling components before an additional nucleotide is added. Still other methods employ the “real-time” detection of incorporation events, where the act of incorporation gives rise to a signaling event that can be detected (See, e.g., Eid, J. et al., Science, 323(5910), 133-138 (2009), hereby incorporated herein by reference). Additional methods for nucleic acid sequence analysis include, but are not limited to, exonuclease sequencing, pyrosequencing, ligase-mediated sequencing, and nanopore-based sequencing. Nanopore-based analysis methods generally involve passing a polymeric molecule, for example single-stranded DNA (“ssDNA”), through a nanoscopic opening while monitoring a signal such as an electrical signal (see, e.g., U.S. Pat. No. 8,986,928, hereby incorporated by reference herein).

Although the cost of generating the newer sequencing information is decreasing and throughput of these technologies and platforms is increasing, it is recognized that focused target enrichment from high complexity nucleic acid samples, e.g., genomic DNA, will improve sequencing at high depth, enabling the sequencing or targeted re-sequencing of a larger number of samples as required for various fundamental biological studies of normal and disease development and pathogenesis.

Various methods for selective enrichment of a multiplicity of targets from genomic DNA, commonly referred to as “genome partitioning,” were developed in recent years. Some of these methods are based on selective hybridization to oligonucleotides designed to hybridize to the user-selected genomic regions. The hybridization can be to oligonucleotides immobilized on high- or low-density microarrays or solution phase hybridization to oligonucleotides modified with a ligand which can be subsequently immobilized to a solid surface, such as a bead. Other methods employ sequence-specific amplification (e.g., PCR) to amplify specific genomic regions in a droplet, allowing clonal amplification of defined regions for downstream sequencing.

There is still a need for improved methods of selectively enriching nucleic acids having a desired target sequence for downstream next-generation applications such as massively parallel sequencing. The present disclosure provides methods and compositions that fulfill this and other needs.

### BRIEF SUMMARY OF THE INVENTION

Disclosed herein are methods and compositions for enriching nucleic acid fragments from a sample that include one or more target region of interest. In certain aspects, a sample of double stranded nucleic acid fragments having at least one strand-linking adapter at one end are denatured, e.g., by heat, and contacted with capture probes specific for a target sequence of interest. Capture probe-bound fragments are then isolated from non-capture probe-bound fragments in the sample, e.g., using a solid substrate specific for the binding moiety on the capture probes, and are renatured for downstream processing. This enrichment process maintains the original double-stranded portion of the nucleic acid fragments that contain the target region of interest in their native states, and thus allows for analysis of epigenetic modifications as well as primary sequence analysis of such fragments. In some aspects, adapter ligated fragments are denatured by initiating nucleic acid synthesis on one strand of the double-stranded nucleic acid fragment insert. In additional aspects, nascent nucleic acid strands generated from the adapter containing fragments are selected by the capture probe and employed for downstream analysis. In some aspects, the nucleic acid fragments have a non-strand linking adapter on the end opposite the strand-linking adapter. In further aspects, the nucleic acid fragments have strand linking adapters at both ends, either the same adapters at both ends (symmetric) or different adapters at each end (asymmetric). The disclosed enrichment process and compositions are suitable for analyzing nucleic acids that are fragmented and/or damaged, e.g., cell-free DNA such as circulating tumor DNA, as well as nucleic acids that are many kilobases in length. Where sequence analysis is performed on the enriched fragments, any suitable method may be employed, including single-molecule sequencing methods (e.g., SMRT® Sequencing or nanopore sequencing).

Specific aspects of the present disclosure include the following.

1. A method of enriching for nucleic acids comprising a target sequence from a mixture of nucleic acids, comprising: providing a mixture of nucleic acids, wherein the nucleic acids comprise: a double-stranded insert region having a first and second end, wherein one or more insert regions include a target sequence; and a strand-linking adapter at the first end; denaturing the double-stranded insert regions of the nucleic acids; contacting the denatured nucleic acids to one or more capture probes comprising a capture region specific for the target sequence, wherein the contacting is under conditions that allow sequence-specific binding of the capture region to the target sequence; isolating nucleic acids bound to the one or more capture probes; removing the one or more capture probes from the isolated nucleic acids; renaturing the double-stranded insert region of the isolated nucleic acids, thereby enriching for nucleic acids comprising the target sequence.

2. The method of aspect 1, wherein the nucleic acids further comprise a second adapter at the second end of the double-stranded insert region.

3. The method of aspect 2, wherein the second adapter is a non-strand-linking adapter.

4. The method of aspect 3, wherein the providing step comprises: obtaining a sample comprising double-stranded DNA fragments; contacting the sample with a mixture of the strand-linking adapter and the non-strand-linking adapter under conditions that allow covalent attachment of the adapters to the ends of the double-stranded DNA fragments.

5. The method of any preceding aspect, wherein the capture region comprises a nucleic acid sequence complementary to one nucleic acid strand of the target region.

6. The method of aspect 5, wherein the nucleic acid sequence in the capture region is an RNA sequence.

7. The method of aspect 6, wherein the removing step comprises contacting the isolated nucleic acids with an RNase that degrades RNA in an RNA/DNA heteroduplex to degrade the capture region RNA sequence.

8. The method of aspect 7, wherein the RNase is RNase H.

9. The method of aspect 5, wherein the nucleic acid sequence in the capture region is a DNA sequence.

10. The method of aspect 9, wherein the removing step comprises contacting the isolated nucleic acids with an exonuclease to degrade the capture region DNA sequence.

11. The method of aspect 10, wherein the exonuclease is selected from the group consisting of: an exonuclease having 3′ to 5′ exonuclease activity on dsDNA; an exonuclease having 5′ to 3′ exonuclease activity on dsDNA; Lambda exonuclease; exonuclease III; and any combination thereof.

12. The method of any preceding aspect, wherein a plurality of capture probes is contacted to the denatured nucleic acids, wherein the plurality of capture probes comprises capture regions that are specific for different target sequences.

13. The method of aspect 12, wherein a first of the plurality of capture probes comprises a capture region specific for a first strand of a first target region and a second of the plurality of capture probes comprises a capture region specific for a second strand of the first target region.

14. The method of any preceding aspect, wherein the strand-linking adapter is a nucleic acid hairpin adapter.

15. The method of aspect 14, wherein the hairpin adapter comprises a nucleic acid synthesis primer binding site.

16. The method of aspect 14 or 15, wherein the hairpin adapter comprises a sequencing primer binding site.

17. The method of aspect 3, wherein the non-strand-linking adapter is a linear nucleic acid adapter.

18. The method of aspect 17, wherein a first end of the linear nucleic acid adapter is configured to ligate to compatible double-stranded DNA ends and the second end of the linear nucleic acid adapter is protected from exonuclease digestion.

19. The method of aspect 18, wherein the linear nucleic acid adapter is protected from exonuclease digestion by the inclusion of phosphorothioate nuclei acid linkages at the second end.

20. The method of aspect 17, 18 or 19, wherein the second end of the linear oligonucleotide adapter comprises a 3′ overhang region that includes a sequencing primer binding site.

21. The method of aspect 17, wherein the linear nucleic acid adapter comprises a restriction enzyme cleavage site, wherein the method further comprises: cleaving the enriched nucleic acids at the restriction enzyme cleavage site; and ligating a second strand-linking adapter to the digested restriction enzyme cleavage site.

22. The method of aspect 21, wherein the second strand-linking adapter is a second hairpin adapter.

23. The method of aspect 22, wherein the second hairpin adapter comprises a sequencing primer binding site.

24. The method of aspect 15, wherein the denaturing comprises: hybridizing a synthesis primer to the nucleic acid synthesis primer binding site in the hairpin adapter of the nuclei acids; and placing the hybridized nucleic acids in a nucleic acid synthesis reaction mixture comprising a strand-displacing nucleic acid polymerase to generate a nascent nucleic acid strand on one strand of the double-stranded nucleic acid insert of the nucleic acids, thereby displacing the complementary strand of the nucleic acids.

25. The method of aspect 24, wherein the nucleic acid synthesis reaction mixture comprises dUTP nucleotides.

26. The method of aspect 24 or 25, wherein the removing and/or renaturing steps comprises contacting the isolated nucleic acids with one or more nucleases that degrade the capture region of the capture probe and the nascent nucleic acid.

27. The method of aspect 25, wherein the one or more nucleases are selected from the group consisting of: an exonuclease that degrades RNA in an RNA/DNA heteroduplex; an exonuclease having 3′ to 5′ exonuclease activity on dsDNA; an exonuclease having 5′ to 3′ exonuclease activity on dsDNA; an exonuclease having 3′ to 5′ exonuclease activity on single stranded DNA; an exonuclease having 5′ to 3′ exonuclease activity on single stranded DNA; an uracil-specific excision reagent (USER); RNase H; Lambda exonuclease; exonuclease I; exonuclease III; and any combination thereof.

28. The method of aspect 27, wherein the USER is a mixture of uracil DNA glycosylase and Endonuclease VIII.

29. The method of any preceding aspect, wherein the one or more capture probes comprise a retrieval region.

30. The method of aspect 29, wherein the retrieval region is a first member of a binding pair.

31. The method of aspect 30, wherein the first member of the binding pair is selected from the group consisting of: a nucleic acid sequence, biotin, avidin, streptavidin, digoxigenin, a protein, an antibody, or combinations thereof.

32. The method of aspect 30 or 31, wherein the isolating step comprises contacting the capture probe-contacted sample with a solid substrate comprising the binding partner of the first member of the binding pair.

33. The method of aspect 32, wherein the solid substrate is a bead.

34. The method of aspect 33, wherein the bead is a magnetic bead and wherein the isolating further comprises: applying a magnetic field to capture the magnetic beads; and washing the captured magnetic beads to remove nucleic acids that are not hybridized to the one or more capture probes.

35. The method of any preceding aspect, further comprising ligating a second strand-linking adapter to the second end of the enriched nucleic acids after the renaturation step.

36. The method of any preceding aspect, further comprising sequencing the enriched nucleic acids.

37. The method of any preceding aspect, wherein the double-strand insert regions of the nucleic acids in the mixture are derived from: genomic DNA, cDNA, cell free DNA, fragmented DNA, damaged DNA, DNA form a formalin-fixed paraffin embedded (FFPE) tissue sample, DNA from a clinical sample, DNA form a tissue sample, and any combination thereof.

38. The method of any preceding aspect, wherein the nucleic acid mixture is a multiplexed sample.

39. The method of aspect 38, wherein the nucleic acids in the multiplexed sample comprise barcodes that allow identification of their source.

40. A kit, comprising: a hairpin adapter comprising a ligation site and a synthesis primer binding site in the loop region; a linear adapter comprising a ligation site at a first end and an exonuclease resistant second end; a ligase; one or more nucleases; a synthesis primer specific for the synthesis primer binding site; a strand-displacing nucleotide polymerase; a solid substrate comprising a first member of a binding pair; and one or more buffers or reagents for performing ligation reactions, nucleic acid synthesis reactions, solid substrate binding reactions, and nuclease reactions.

41. The kit of aspect 40, wherein the one or more nucleases are selected from the group consisting of: an exonuclease that degrades RNA in an RNA/DNA heteroduplex; an exonuclease having 3′ to 5′ exonuclease activity on dsDNA; an exonuclease having 5′ to 3′ exonuclease activity on dsDNA; an exonuclease having 3′ to 5′ exonuclease activity on single stranded DNA; an exonuclease having 5′ to 3′ exonuclease activity on single stranded DNA; an uracil-specific excision reagent (USER); RNase H; Lambda exonuclease; exonuclease I; exonuclease III; and any combination thereof.

42. The kit of aspect 40 or 41, wherein the strand displacing polymerase is selected from the group consisting of: a Φ29 DNA polymerase or modified version thereof, a homolog of a Φ29 DNA polymerase or modified version thereof, and combinations thereof.

43. The kit of any one of aspects 40 to 42, wherein the solid substrate is a bead and the first member of the binding pair is selected from the group consisting of: a nucleic acid sequence, biotin, avidin, streptavidin, digoxigenin, a protein, an antibody, or combinations thereof.

### BRIEF DESCRIPTION OF THE DRAWINGS

FIG. 1 shows a first embodiment for enriching a nucleic acid fragment having a target sequence of interest from a mixture of nucleic acids.

FIG. 2 shows a second embodiment for enriching a nucleic acid fragment having a target sequence of interest from a mixture of nucleic acids.

FIG. 3 shows a third embodiment for enriching a nucleic acid fragment having a target sequence of interest from a mixture of nucleic acids.

FIG. 4 shows a fourth embodiment for enriching a nucleic acid fragment having a target sequence of interest from a mixture of nucleic acids.

FIG. 5 shows a fifth embodiment for enriching a nucleic acid fragment having a target sequence of interest from a mixture of nucleic acids.

FIG. 6 shows a sixth embodiment for enriching a nucleic acid fragment having a target sequence of interest from a mixture of nucleic acids.

FIG. 7 shows a seventh embodiment for enriching a nucleic acid fragment having a target sequence of interest from a mixture of nucleic acids.

### DETAILED DESCRIPTION OF THE INVENTION

As summarized above, the present disclosure provides methods and compositions for enriching nucleic acid fragments having a target region (or target sequence) of interest from a mixed sample, e.g., a library of nucleic acids. In some embodiments, multiple different target regions are enriched from a sample using multiple different capture probes. In many embodiments, the enriched nucleic acids are maintained in their native state, allowing for downstream analyses of not only their primary sequence but also any epigenetic modifications.

In general, enrichment involves removing desired nucleic acid species (i.e., those that have a target region/sequence of interest) from a mixture of other components, including nucleic acid species that do not include the target region/sequence of interest.

The methods and compositions of the invention are directed to isolating double-stranded nucleic acids from a sample of nucleic acids (e.g., a nucleic acid library) where the double-stranded nucleic acids in the sample have at least one strand-linking adapter at one end and where the method includes: opening up the double-stranded region to expose a sequence within the region, contacting the exposed sequence with a capture probe specific for a region/sequence of interest under appropriate capture probe/target region binding conditions (e.g., hybridization conditions); and isolating capture-probe bound nucleic acids. The isolated nucleic acids can be subjected to any desired downstream process, e.g., removal of the capture probe, renaturation, additional adapter attachment, and/or sequence analysis.

The methods and compositions can be used to selectively enrich for nucleic acids having specific sequences from a mixture of nucleic acids. For example, for DNA sequencing, DNA fragments from any desired source (e.g., circulating cell-free DNA or isolated and fragmented genomic DNA) can be treated, e.g. by ligation, to attach a strand-linking adapter to at least one end of the fragments. These strand-linking adapters function not only to keep the complementary strands of the DNA fragments together during the enrichment process, but also can be used to append sites for priming, for formation of polymerase-nucleic acid complexes, for barcoding, etc., as desired. With the methods described herein, capture probes can be used to selectively enrich for fragments containing desired target sequences.

Throughout the application, either the term enrichment or the term removal is used to mean separating a component from other components in a mixture. For example, in some cases there is removal of the capture oligonucleotide by a bead. The removal of the capture oligonucleotide results in isolation of the compound to which the capture oligonucleotide is attached.

Aspects of the present disclosure are described in further detail below.

### Nucleic Acids and Adapters

A double-stranded nucleic acid sample that can be subjected to the enrichment processes as described herein can be obtained in any convenient manner. In certain embodiments, the nucleic acid sample is obtained in a form that is amenable to enrichment, i.e., it has already been processed such that it includes double-stranded DNA fragments having at least one strand-linking adapter attached. In other embodiments, the methods include attaching one or more adapters to double-stranded DNA fragments, e.g., via ligation (described further below). The double-stranded nucleic acids to be enriched can be from any desired source, and as such no limitation in this regard is intended. In certain embodiments, the source of the nucleic acids selected from a tissue sample, a body fluid, a cell sample, or a stool sample. In certain embodiments, the source is a body fluid, such as whole blood, saliva, tears, sweat, sputum, or urine. In some cases, only a portion of the whole blood, such as blood plasma or cell free nucleic acid is used. In other cases, the source is a tissue sample, such as a formalin-fixed paraffin-embedded (FFPE) tissue sample, a fresh frozen (FF) tissue sample, or a combination thereof.

In certain embodiments, the parent nucleic acid sample is a sample of cell free DNA (cfDNA), which are short nuclear-derived DNA fragments present in a bodily fluids (e.g., plasma, stool, urine) (see, e.g., Mouliere and Rosenfeld, 2015, “Circulating tumor-derived DNA is shorter than somatic DNA in plasma”, PNAS 112(11): 3178-3179; Jiang et al., 2015, “Lengthening and shortening of plasma DNA in hepatocellular carcinoma patients”, PNAS 112(11):E1317-25; and Mouliere et al., 2014, “Multi-marker analysis of circulating cell-free DNA toward personalized medicine for colorectal cancer”, Molecular Oncology, 8(5):927-41; hereby incorporated by reference herein in their entireties). Tumor derived circulating tumor DNA (ctDNA) constitutes a minority population of cfDNA, varying up to about 50%. In some embodiments, ctDNA varies depending on tumor stage and tumor type. In some embodiments, ctDNA varies from about 0.001% up to about 30%, such as about 0.01% up to about 20%, such as about 0.01% up to about 10% of cfDNA. The covariates of ctDNA are not fully understood, but appear to be positively correlated with tumor type, tumor size, and tumor stage (see, e.g., Bettegowda et al, 2014 “Detection of circulating tumor DNA in early- and late-stage human malignancies”, Sci Trans Med, 6(224):224; and Newmann et al, 2014, “An ultrasensitive method for quantitating circulating tumor DNA with broad patient coverage”, Nature Medicine, 20(5):548-54; both hereby incorporated by reference herein in their entireties). Despite the challenges associated with the low population of ctDNA in cfDNA, tumor variants have been identified in ctDNA across a wide span of cancers. Furthermore, analysis of cfDNA versus tumor biopsy is less invasive and methods for analyzing, such as sequencing, enable the identification of sub-clonal heterogeneity.

In some embodiments, and prior to adapter attachment, the starting DNA is derived from a source from which the DNA is already in a fragmented form, e.g., cfDNA or DNA from forensic or pathology specimens. In other embodiments, the starting DNA is in a form that is subjected to a fragmentation process, e.g., a genomic DNA sample that is fragmented in any suitable manner, e.g., by enzymatic, chemical, and/or physical means, including shearing or restriction enzyme fragmentation. Regardless of their original state, DNA fragments can be treated to repair damage and/or produce ends that are amenable to further processing. For example, DNA derived from Formalin-Fixed, Paraffin-Embedded (FFPE) samples can be treated with the NEBNEXT® FFPE DNA Repair Mix, which is a cocktail of enzymes formulated to repair DNA, and specifically optimized and validated for repair of FFPE DNA samples. In some embodiments, enzymes can be added that produce DNA ends that are suitable for ligation to adapters, e.g., to create blunt ends or ends with nucleotide overhangs (5′ or 3′). Numerous such DNA repair and end modification methods are known in the art and can be employed as desired by a user.

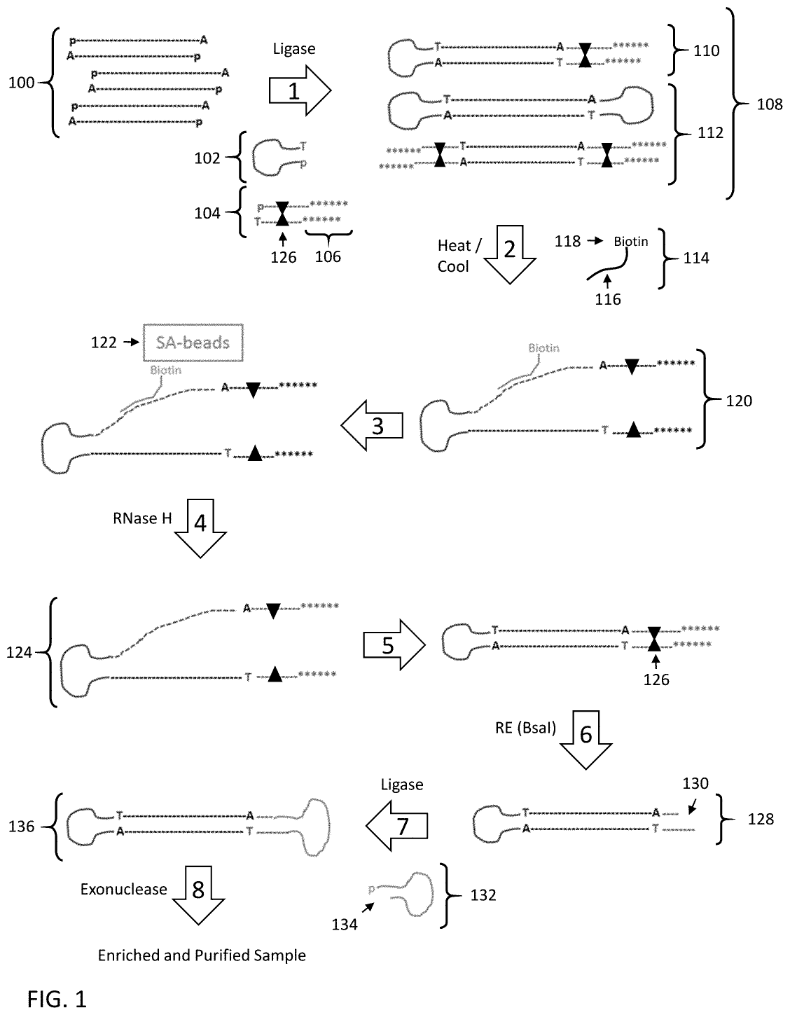

As noted above, nucleic acid samples that are used in the disclosed enrichment methods described herein include double-stranded DNA fragments (sometimes referred to as DNA inserts) having at least one strand-linking adapter attached. By “strand-linking” is meant that the adapter functionally links the 5′ end of a first strand of a DNA insert to the 3′ end of the complementary strand of the DNA insert. This link needs to be sufficiently stable to allow the strands of the double-stranded DNA insert to remain attached to one another, via the strand-linking adapter, under the conditions of the enrichment process that result in the separation of the two strands of the DNA insert, as when the strands are separated to allow for binding of the capture probe to its cognate binding site (described below). Any adapter that achieves this may be used. In certain embodiments, strand-linking adapters covalently link the 5′ end of one strand of a DNA insert to the 3′ end of the complementary strand. Any type of covalent linkage can be used, but are not limited to, a polymeric linker, a chemical linker, a polynucleotide or a polypeptide. In some embodiments, the strand-linking adapter comprises DNA, RNA, modified DNA (such as abasic DNA), RNA, PNA, LNA, or PEG. In some embodiments, the bridging moiety is an oligonucleotide-based hairpin adaptor. In certain embodiments, hairpin adapters are oligonucleotides that have both a single stranded loop region and a double stranded region that forms a site that is designed for attachment to the end of a double-stranded DNA insert, e.g., having a blunt end or a nucleotide overhang that is compatible with a nucleotide overhang on the double stranded DNA ends. An example of a hairpin adaptor is shown in FIG. 1, element 102. Hairpin adapters may include non-nucleotide covalent linkages as well, including PEG, PNA, and/or PEG linkages, for example. No limitation in this regard is intended. In additional embodiments, the strand-linking adapter links the strands of the DNA insert non-covalently using members of a binding pair, e.g., avidin/biotin, antibody (or binding fragment thereof)/antigen, receptor/ligand, etc. (See description of binding members below.)

In some embodiments, double-stranded DNA fragments that are subject to the enrichment processes described herein have a strand-linking adapter (a first adapter) at a first end and a second adapter at the second end. The second adapter can be identical to the first adapter, called symmetrically-tagged DNA fragments, or alternatively the second adapter can be different from the first adapter, called asymmetrically-tagged DNA fragments. Such different second adapters have at least one difference form the first adapter, e.g., at least one different nucleotide, nucleotide sequence, moiety, or modification, as desired by the user. In some embodiments, the second adapters is a strand-linking adapter while in other embodiments the second adapter is not a strand-linking adapter, sometimes referred to herein as a linear adapter. As the name implies, linear adapters do not link the two strands of a DNA fragment together. In general, linear adapters are oligonucleotide containing species that, similar to strand-linking adapters, include a double-stranded region that forms a site that is designed for attachment to the end of a double-stranded DNA insert, e.g., having a blunt end or a nucleotide overhang that is compatible with a nucleotide overhang on the double stranded DNA ends. Linear adapters may also include single stranded regions, e.g., 5′ overhangs, 3′ overhangs, Y regions, bubble regions (a non-complementary region flanked by complementary double-stranded regions), or any combination thereof, as desired by a user. See FIG. 1 for one example of a linear adapter (element 104).

The terms “nucleic acid” or “oligonucleotide” or grammatical equivalents thereof mean at least two nucleotides covalently linked together. A nucleic acid will generally contain phosphodiester bonds, e.g., as found in naturally occurring DNA and RNA. However, in some cases, nucleic acid analogs are included in this general description of nucleic acids or oligonucleotides. For example, oligonucleotides that are used as adapters or primers may have alternate backbones, comprising, for example, phosphoramide, phosphorothioate, phosphorodithioate, and peptide nucleic acid backbones and linkages. Other analog nucleic acids include those with positive backbones, non-ionic backbones, and non-ribose backbones, including those described in U.S. Pat. Nos. 5,235,033 and 5,034,506. The nucleic acids may also have other modifications, such as the inclusion of heteroatoms, the attachment of labels, such as dyes, or substitution with functional groups which will still allow for base pairing and for recognition by the enzyme. Both strand linking and linear adapters can include any other functional sequences, domains, regions, and/or moieties as desired by a user and/or that find use in certain downstream process or analysis steps, e.g., as described herein. Examples of such include primer binding sites (e.g., for sequencing primer binding), universal capture probe binding sites, barcode sequences, restriction enzyme sites, special/modified nucleotides or nucleotide linkages (e.g., exonuclease-resistant nucleotides), binding moieties, site or structure designed for enzyme binding (e.g., helicase binding as employed in nanopore sequencing; see, e.g., U.S. Patent Application Publication No. US2015/0152492 entitled “Hairpin loop method for double strand polynucleotide sequencing using transmembrane pores” which is hereby incorporated by reference herein in its entirety), and the like. No limitation in this regard is intended. For example, a universal nucleic acid synthesis primer binding site can be included that allows a single primer to initiate nucleic acid synthesis on all of the adapter-ligated fragments even though the fragments can have double-stranded DNA inserts having different sequences. In addition, regions in the adapters can act as a universal capture sequences for the enrichment of any nucleic acids that have this portion of the adaptor, regardless of the sequence of the DNA insert.

As noted above, nucleic acids for enrichment according to the present disclosure can be derived from any suitable natural or synthetic source. While in certain embodiments, the nucleic acid comprises double stranded DNA, in some circumstances double-stranded RNA or RNA-DNA heteroduplexes can be used. Any minor alterations to the methods and compositions of the present disclosure that are needed to process such alternative nucleic acids can be envisioned by the ordinarily skilled artisan.

### Strand Separation

In aspects of the present disclosure, once the desired double-stranded nucleic acids with at least one strand-linking adapter are obtained, the strands of the insert are separated, which exposes a region of interest in at least one strand for capture probe binding (detailed below). Separation can be achieved in any convenient manner, including placing the double-stranded nucleic acids under denaturing conditions, e.g., by heat or chemical treatment, or by using the strand displacing activity of an enzyme, e.g., a strand-displacing nucleic acid polymerase or a helicase.

In embodiments that employ a strand-displacing nucleic acid polymerase, a synthesis primer binding site present in one of the adapters, e.g., in the single-stranded region of a strand-linking oligonucleotide hairpin adapter, can be used as a site to initiate nucleic acid synthesis. This generally entails hybridizing a synthesis primer to the synthesis primer binding site and contacting with a strand displacing nucleic acid polymerase under nucleic acid synthesis conditions. The polymerase will use one strand of the nucleic acid insert as the template for nucleic acid synthesis, thereby producing a complementary nascent nucleic acid strand, while displacing the other strand and rendering it open for binding by a capture probe. Any convenient strand displacing nucleic acid polymerase for use in such strand separation steps can be used, including wild-type or engineered polymerases, e.g., point mutants, truncated, and/or chimeric polymerase molecules.

DNA polymerase enzymes have also been modified in any of a variety of ways, e.g., to reduce or eliminate exonuclease activities (many native DNA polymerases have a proof-reading exonuclease function that interferes with, e.g., sequencing applications), to simplify production by making protease digested enzyme fragments such as the Klenow fragment recombinant, etc.

In certain embodiments, the strand-displacing polymerase is a Φ29-type DNA polymerase or variant thereof having desired functional characteristics. In one aspect, the polymerase that is modified is a Φ29-type DNA polymerase. For example, the modified recombinant DNA polymerase can be homologous to a wild-type or exonuclease deficient Φ29 DNA polymerase, e.g., as described in U.S. Pat. Nos. 5,001,050, 5,198,543, or 5,576,204, hereby incorporated by reference herein in their entireties. Alternately, the modified recombinant DNA polymerase can be homologous to other Φ29-type DNA polymerases, such as B103, GA-1, PZA, Φ15, BS32, M2Y, Nf, G1, Cp-1, PRD1, PZE, SF5, Cp-5, Cp-7, PR4, PR5, PR722, L17, Φ21, or the like. For nomenclature, see also, Meijer et al. (2001) “Φ29 Family of Phages” Microbiology and Molecular Biology Reviews, 65(2):261-287, hereby incorporated by reference herein in its entirety. Suitable polymerases are described, for example, in U.S. Pat. Nos. 8,420,366 and 8,257,954, hereby incorporated by reference herein in their entireties.

### Conditions for Nucleic Acid Synthesis

The conditions required for nucleic acid synthesis are well known in the art. The polymerase reaction conditions include the type and concentration of buffer, the pH of the reaction, the temperature, the type and concentration of salts, the presence of particular additives that influence the kinetics of the enzyme, and the type, concentration, and relative amounts of various cofactors, including metal cofactors.

Enzymatic reactions are often run in the presence of a buffer, which is used, in part, to control the pH of the reaction mixture. Buffers suitable for the invention include, for example, TAPS (3-{[tris(hydroxymethyl)methyl]amino}propanesulfonic acid), Bicine (N,N-bis(2-hydroxyethyl)glycine), TRIS (tris(hydroxymethyl)methylamine), ACES (N-(2-Acetamido)-2-aminoethanesulfonic acid), Tricine (N-tris(hydroxymethyl)methylglycine), HEPES 4-2-hydroxyethyl-1-piperazineethanesulfonic acid), TES (2-{[tris(hydroxymethyl)methyl]amino}ethanesulfonic acid), MOPS (3-(N-morpholino)propanesulfonic acid), PIPES (piperazine-N,N′-bis(2-ethanesulfonic acid)), and MES (2-(N-morpholino)ethanesulfonic acid).

The pH of the reaction can influence the rate of the polymerase reaction. The temperature of the reaction can be adjusted to enhance the performance of the system. The reaction temperature may depend upon the type of polymerase which is employed.

As used in the art, the term nucleotide refers both to the nucleoside triphosphates that are added to a growing nucleic acid chain in the polymerase reaction, and also to refer to the individual units of a nucleic acid molecule, for example the units of DNA and RNA. Herein, the term nucleotide is used consistently with its use in the art. Whether the term nucleotide refers to the substrate molecule to be added to the growing nucleic acid or to the units in the nucleic acid chain can be derived from the context in which the term is used. The nucleotides or set of nucleotides used during nucleic acid synthesis are generally naturally occurring nucleotides but can also include modified nucleotides (nucleotide analogs). The nucleotides used in the invention, whether natural, unnatural, modified or analog, are suitable for participation in the polymerase reaction.

### Capture Probes

Once the strands of the DNA insert have been separated, one or more capture probes are added that are each specific for a desired target region of interest. In certain embodiments, the capture probe has at least two portions or regions: (1) a capture region and (2) a retrieval region. The capture region is designed to bind specifically to a particular sequence in the DNA insert portion of a template nucleic acid that is exposed upon strand separation. The retrieval region allows the capture probe to be removed from other components of the mixture along with any templates to which it is bound. The capture region can be directly connected to the retrieval region, or the capture probe can have an intermediate region connecting the capture and retrieval regions. The connection between the capture region and the retrieval region can be made with any suitable linkage, whether covalent or non-covalent.

In certain embodiments, the capture region comprises an oligonucleotide with a region complementary to a sequence on the template nucleic acid that is exposed when the strands are separated. Where a capture oligonucleotide is used, the length of the capture region can vary depending on the application. It is well known that the strength and selectivity of binding of complementary or partly complementary oligonucleotides can be controlled by controlling the stringency of the medium, including the ionic strength of the solution and the temperature. The capture region will generally be designed both to have efficient and specific binding as well as reversible binding, allowing for separation of the capture probe from its bound (or “captured”) template after isolation. In some cases, the length of the capture oligonucleotide on the probe is from about 10 to about 200 nucleotides, from about 20 to about 100 nucleotides, or from about 30 to about 50 nucleotides in length. A capture region can comprise natural nucleotide units, non-natural nucleotide units, e.g. PNA, or any combination thereof.

The capture region can also comprise other suitable molecules that specifically bind to an exposed sequence on the nucleic acid. For example, the capture region can comprise transcription factors, histones, antibodies, nucleic acid binding proteins, and nucleic acid binding agents, etc., that will bind to a specific sequence (see, e.g., Blackwell et al. Science 23 Nov. 1990:Vol. 250, 1149-1151 and Kadonaga et al. PNAS, 83, 5889-5893, 1986, and Ren et at. Science, 290, 2306-2309, 2000; hereby incorporated by reference herein in their entirety). The capture region can comprise an antibody that is designed to attach to a specific sequence (see, e.g., LeBlanc et al., Biochemistry, 1998, 37 (17), pp 6015-6022, hereby incorporated by reference herein in its entirety). In some cases, the capture region can comprise agents that will specifically bind regions of the template nucleic acid template that have modified or unnatural nucleotide. For example, antibodies against 5-MeC are used to enrich for methylated DNA sequences (see, e.g., M. Weber, et al., Nat. Genet. 2005, 37, 853, hereby incorporated by reference herein in its entirety). In certain embodiments, the modification is an 8-oxoG lesion and/or the agent is a protein is selected from the group consisting of hOGG1, FPG, yOGG1, AlkA, Nth, Nei, MutY, UDG, SMUG, TDG, or NEIL. In other embodiments, the modification is a methylated base and/or the agent is a protein selected from the group consisting of MECP2, MBD1, MBD2, MBD4, and UHRF1 (see, e.g., U.S. Patent Application Publication No. US2011/0183320 entitled “Classification of nucleic acid templates”, hereby incorporated by reference herein in its entirety). In certain embodiments, a capture probe contains a variant CRISPR/Cas9 protein that lacks nuclease activity complexed with a target-specific guide RNA. Such mutant complexes specifically bind to, but do not cut, their cognate target sequence (see, e.g., PCT Application Publication WO2016/014409 entitled “Polynucleotide enrichment using crispr-cas systems” and U.S. Patent Application Publication No. US2014/0356867 entitled “Nucleic acid enrichment using cas9”, each of which are hereby incorporated by reference herein in their entirety). Capture probes may contain other engineered sequence-specific binding proteins/domains, including those containing transcriptional activator-like effector domains (TALE domains) (see, e.g., U.S. Pat. No. 9,359,599 entitled “Engineered transcription activator-like effector (TALE) domains and uses thereof”, hereby incorporated by reference herein in its entirety).

It is emphasized here that any single capture probe or combination of capture probes may be employed as desired by a user. In some cases, a single type of capture probe comprising a single type of capture region is used whereas in other cases, a mixture of different types of capture probes is used in which each type of capture probe has a capture region directed at a different sequence. The mixtures of capture probes are generally used for isolating (or enriching for) nucleic acids having specific sequences from a population of nucleic acids (where the population comprises nucleic acids that include the specific sequences mixed with nucleic acids that do not contain the specific sequences). This method could be directed to pulling down all conserved sequences of genes from a genetic pathway, derived from one organism, but targeted at a second distinct organism. Alternatively, a family of genetic homologs, orthologs and/or paralogs could be targeted for conservation testing. Alternatively, forensic DNA sequencing (e.g., for crime scene investigation) may target a handful of unique identifying sequences in specific loci including, e.g., unique short tandem repeats, which can enable the confident identification of individuals. The number of different capture probes, each targeting a different sequence, can be from about 2 to about 100,000 or more. In some cases, mixtures have from about 5 to about 10,000 or from about 10 to about 1000 different capture regions. The isolation of specific nucleic acid sequences of interest is valuable when greater efficiency of characterization is desired. For example, even with current sequencing technologies, sequencing of whole genomes for multiple individuals can be impractical. However, by focusing on specific regions of interest, characterization of multiple genomes can be made more practical (see, e.g., Teer J K, Mullikin J C. “Exome sequencing: the sweet spot before whole genomes”, Human Molecular Genetics. 2010 Oct. 15; 19(R2):R145-51 and Mamanova L, Coffey A J, Scott C E, Kozarewa I, Turner E H, Kumar A, Howard E, Shendure J, Turner D J. “Target-enrichment strategies for next-generation sequencing” Nature Methods. 2010 February; 7(2):111-8; hereby incorporated by reference herein in their entireties).

In some cases, two or more capture probes are employed for a region of interest where the capture region of each capture probe targets the same strand of the double-stranded portion of the capture region. In such cases, the capture probes can be designed to not interfere with each other for binding to the region of interest (e.g., they bind to non-overlapping sequences in the region of interest). In some cases, two or more capture probes are employed for a region of interest where the capture region of a first of the capture probes targets one strand, and the capture region of a second of the capture probes targets the complementary strand.

In some cases, in order to capture larger nucleic acid sequences, tiling strategies can be used, whereby sets of capture probes with shorter oligonucleotide capture regions are used with each member of the set targeted to a different portion of the larger nucleic acid sequence. For example, in some cases it could be desired to specifically target a 2 kb sequence of DNA within a library generated by fragmenting genomic DNA. Any given fragment may only have a portion of the 2 kb sequence of interest, so in order to capture such portions, capture probes with oligonucleotide capture regions designed to bind to various different portions of the 2 kb sequence can be provided. For example, a tiling strategy could be employed in which a set of capture oligonucleotides was provided for targeting on average, each 50 base region along the 2 kb sequence. This would result in a set of about 40 capture oligonucleotides. The nucleic acid portion which is tiled for capture could be from about 100 bases to greater than 1000 kb long. In some cases, it could be between about 1 kb and about 100 kb. The average sequence for each tile can be varied as needed for the application, and could range, for example, from about 20 bases to about 500 bases. The number of capture sequences directed at a nucleotide sequence can be, for example, from about 10 to about 1000, or from about 20 to about 200. The tiled capture sequences can be used to selectively capture and isolate desired sets of sequences. For example, in some cases, a specific exon, or a specific family of exons could be targeted for isolation. The exons of a specific organism such as human or mouse could be targeted. In some cases, the nucleic acids characteristic of a specific virus, bacterium, or pathogen or a specific strain can be targeted. In other cases, nucleic acids representing various functional classes, e.g. those coding for kinases can be targeted for isolation. In some cases, nucleic acids of interest in a particular biological process, such as those implicated in cancer progression or response to drug therapies, can be targeted.

In some cases, an iterative capture and retrieval process is employed where a first capture oligonucleotide targeting a first sequence is used to isolate nucleic acids having the that sequence, then in a subsequent step, a second capture oligonucleotide is used to capture a second sequence. This results in the enrichment of nucleic acids having both the first and the second sequences of interest. In some cases, the first and second sequences are on the same strand of the double stranded portion of the nucleic acid, and in some cases one sequence is on one strand of the nucleic acid and the other sequence is on the other strand. In some cases, rather than a single first capture oligonucleotide, a set of first capture oligonucleotides are used to capture a set of first nucleic acids. Analogously, in some cases, rather than a second oligonucleotide, a set of second oligonucleotides is used to capture a set of second nucleic acids. These iterative isolation and purification methods allow for selecting and isolating only complexes having a desired set of sequences.

In some embodiments, the capture probe comprises beads that have two types of capture regions attached to them, a first capture region directed to a first sequence, and a second capture region directed to a second sequence. These capture beads are added to a solution with a mixture of template nucleic acids, some having only the first or the second capture sequence, and some having both the first and the second capture sequence. The stringency of the solution is adjusted such that nucleic acids bound to only one of the capture regions will be washed off, but nucleic acids bound through both the first capture region and the second capture region will remain bound to the beads. This provides a one-step method for isolating nucleotides from the mixture that have two sequences of interest. In some cases, the two sequences are on the same strand; in some cases, the two sequences are on opposite strands. While this approach is generally used with two types of capture regions on a bead, the same approach can be used employing beads having 3, 4, or more types of capture regions attached to them, but the difficulty of controlling the hybridization to differentiate the multiply-bound species goes up with the number of different capture regions.

The retrieval region of the capture probe is provided for removal and isolation of capture probe/nucleic acid complexes, i.e., a capture probe and a cognate nucleic acid bound to the capture region (where the bound nucleic acid may also be bound to additional components, e.g., a polymerase, a nascent nucleic acid strand, or both). In some cases, the retrieval region comprises a bead or other solid surface. In some cases, the retrieval region comprises a member of a binding pair which allows for removal of the capture probe and any complexed nucleic acid by a bead or surface comprising the other member of the binding pair. The binding pair for retrieval of the capture probe can bind by hybridization, ionic, H-bonding, VanderWaals or any combination of these forces. In some cases, the retrieval can be done using hybridization, e.g. using specific sequences or by using polynucleotide sequences. For example, one member of the biding pair can comprise either poly(A), poly(dA), poly(C) or poly(dC), and the other binding member can comprise poly(T), poly(dT), poly(G) or poly(dG). The length of the polynucleotide sequence can be chosen to provide the best binding and release properties. The binding and release can be controlled, for example, by controlling the stringency of the solution. Non-natural and modified bases can also be used in order to control the binding and release properties.

Binding pair members can comprise, e.g., biotin, digoxigenin, inosine, avidin, GST sequences, modified GST sequences, e.g., that are less likely to form dimers, biotin ligase recognition (BiTag) sequences, S tags, SNAP-tags, enterokinase sites, thrombin sites, antibodies or antibody domains, antibody fragments, antigens, receptors, receptor domains, receptor fragments, or combinations thereof.

The use of beads for isolation is well known in the life sciences, and any suitable bead isolation method can be used with the present invention. As described above, the beads can be part of the capture probe, or can be added in a subsequent step to bind to and retrieve the capture probe and any complexed nucleic acids. Beads can be useful for isolation in that molecules of interest can be attached to the beads, and the beads can be washed to remove solution components not attached to the beads, allowing for purification and isolation. The beads can be separated from other components in the solution based on properties such as size, density, or dielectric, ionic, and magnetic properties. In preferred embodiments, the beads are magnetic. Magnetic beads can be introduced, mixed, removed, and released into solution using magnetic fields. Processes utilizing magnetic beads can also be automated. Magnetic beads are supplied by a number of vendors including NEB, Dynal, Micromod, Turbobeads, and Spherotech. The beads can be functionalized using well known chemistry to provide a surface having the binding groups required for binding to the capture probe.

Solid surfaces other than beads can also be used to retrieve the capture probes nucleic acids attached. The solid surfaces can be planar surfaces, such as those used for hybridization microarrays, or the solid surfaces can be the packing of a separation column.

Multiple specific capture probes can be added where it is desired to isolate nucleic acids having any one of a set of target sequences, each different capture probe specific for a different target sequence. In some embodiments, all or a subset of the nucleic acid sequences that form the target-binding regions in the capture probes can be overlapping and/or target complementary strands of the target sequences of interest. No limitation in the design of target-binding regions by a user in mixtures of different capture probes is intended.

In some cases, capture probes can be made to target sequences that are not desired, e.g. for background knockdown. Such use of capture probes can be referred to as negative selection/enrichment (enrichment of nucleic acids from a sample that do not do not bind to capture probes specific for undesired target sequences). There are situations, for example in DNA sequencing, in which there are contaminating sequences that are not desired and will use up useful sequencing resources. For example, in some cases, capture probes can be used to target sequences representing housekeeping genes in order to remove these from the mixture. Thus, in some embodiments, capture probes for capturing both desired and undesired sequences will be deployed, with the undesired sequences separated from those desired. This can be done sequentially, e.g. by first exposing the sample to capture probes specific for the undesirable sequences, removing those beads from the sample, then in a second step exposing the supernatant of the first step to capture probes specific for the desired sequences. In some cases, capture probes specific for the desired and undesired sequences can be added at the same time. For example, the capture probes specific for undesired sequences can be attached to non-magnetic beads, and the capture probes specific for desired sequences attached to magnetic beads, allowing for selective removal or isolation of only the desired sequences by magnetic isolation.

### Isolation/Purification

The nucleic acid, and any associated moieties (e.g., polymerases or nascent nucleic acid strands), that is bound to the capture probe and retrieved can then be isolated and purified to form an enriched sample (a sample enriched for nucleic acids having a target sequence of interest). Where the capture probe is bound to a solid surface such as a bead, planar surface, or column, fluid can be washed over the solid surface, removing components of the original mixture that are not bound to the solid surface, leaving behind on the surface the attached capture probe/nucleic acid complex. This washing can remove, for example, inactive polymerase-nucleic acid complex, excess enzyme, unbound nucleic acids and other components. The wash fluid will generally contain components that assist in maintaining the stability of the capture probe/nucleic acid complex, e.g. by maintaining levels of specific ions, the required level of ionic strength, and the appropriate pH. The stringency of the medium is also controlled during the wash to ensure that the capture probe/nucleic acid complex remains bound during the wash. In certain embodiments, the stringency of the binding and wash media are designed to maintain binding of polymerases and/or nascent strands to the nucleic acid in the capture probe/nucleic acid complex.

### Sequencing of Enriched Nucleic Acids

In certain aspects of the invention, the enriched nucleic acids are subjected to sequence analysis. As indicated above, one benefit of the present disclosure is direct sequencing of enriched original nucleic acids from a sample of interest, and not enriched amplified products of the original nucleic acids. This not only reduces the introduction of sequence errors during amplification, but also allows analysis of epigenetic modification of the original nucleic acid molecules.

The amplified nucleic acids produced can serve as sequencing templates in many different types of sequencing systems, e.g., Sanger sequencing systems, capillary electrophoresis systems, Ion Torrent™ systems (Life Technologies), and MiSeq® and HiSeq® systems (Illumina, Inc.). Preferably, such sequence analysis is performed using a technology that can produce sequence reads from single template molecules, such as nanopore-based sequencing, e.g., from Oxford Nanopore or Genia Technologies. One particularly preferred single-molecule sequencing technology is SMRT® Sequencing from Pacific Biosciences (Menlo Park, Calif.), which is described in detail in the art, e.g., in U.S. Pat. Nos. 7,056,661, 6,917,726, 7,315,019, and 8,501,405; Eid, et al. (2009) Science 323:133-138; Levene, et al. (2003) Science 299:682-686; Korlach, et al. (2008) Nucleosides, Nucleotides and Nucleic Acids 27:1072-1083; and Korlach, et al. (2010) Methods in Enzymology 472:431-455, all of which are hereby incorporated by reference herein in their entireties for all purposes. Briefly, SMRT® Sequencing is a real-time sequencing method in which a single polymerase-template complex is observed during template-directed synthesis of a complementary nascent strand. Unlike conventional “flush-and-scan” sequencing methods, the SMRT® Sequencing reaction involves processive strand synthesis by the polymerase, without the need for buffer exchange in between successive base incorporation events. Nucleotide analogs present in the sequencing reaction mixture comprise optically detectable labels (typically fluorescent dyes), which are linked to the analogs at a phosphate group that is removed during incorporation of the nucleoside portion into the nascent strand. As such, the nascent strand produced is “natural” and contains no fluorescent dyes, which diffuse away into the reaction mixture after the incorporation event. During the reaction, the polymerase-template complex is immobilized in an optical confinement called a “zero-mode waveguide” that significantly reduces the background fluorescence to facilitate detection of individual incorporation events. Since SMRT® Sequencing produces sequence reads from a single template molecule, the presence of a barcode, e.g., in an attached adapter, allows individual sequence reads to be correlated to a single, parental nucleic acid molecule.

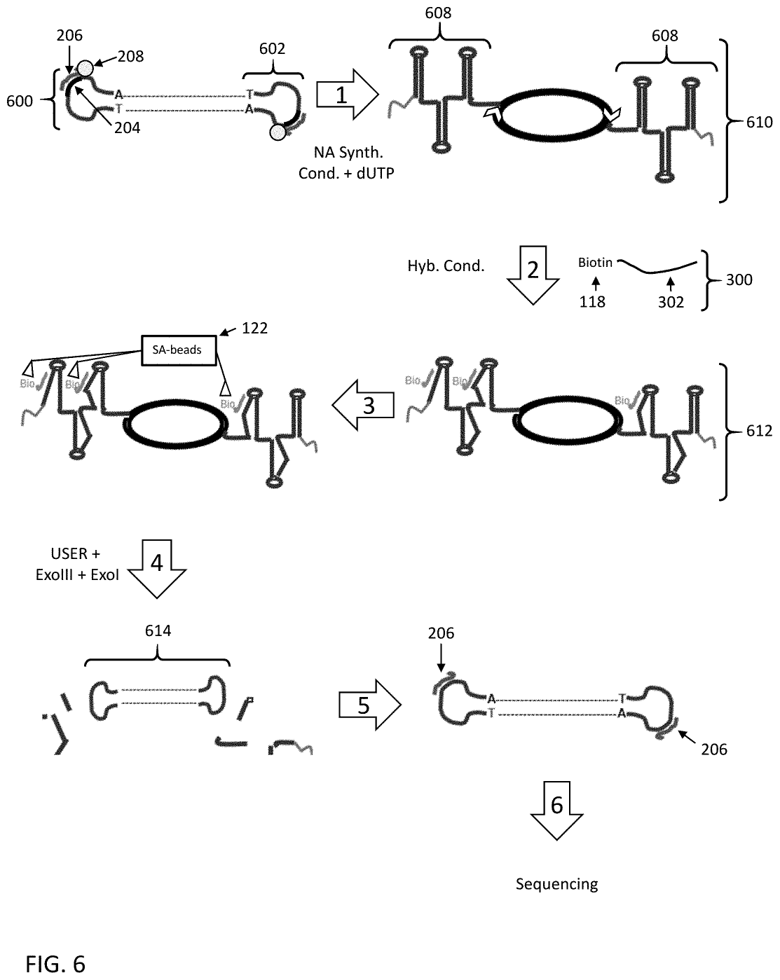

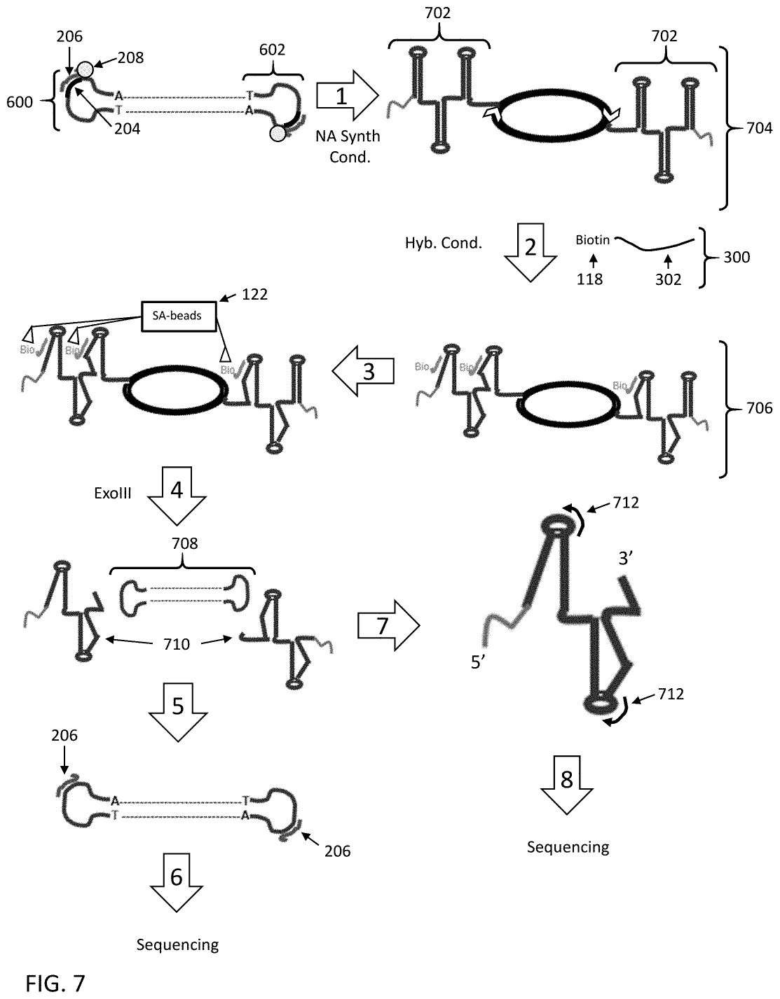

In some embodiments, the nucleic acids that are subjected to enrichment (e.g., adapter attached nucleic acids in the nucleic acid library) are in a cyclic form (e.g., SMRTBELL® templates, e.g., as in FIGS. 6 and 7). In other embodiments, the enriched nucleic acids are converted to a cyclic form after enrichment (e.g., as in FIGS. 1, 2, and 3). Performing single-molecule sequencing, e.g., SMRT® Sequencing or nanopore sequencing (e.g., using rolling circle replication-based methods as described in U.S. Pat. No. 9,494,554, entitled “Chip set-up and high-accuracy nucleic acid sequencing” which is hereby incorporated by reference herein in its entirety), on a cyclic nucleic acid template is advantageous in that it allows for redundant sequencing of a given region. The accuracy of a sequence determination can be improved significantly by sequencing the same region multiple times. Cyclic nucleic acids that are highly useful for the current invention include SMRTBELL® templates, which are nucleic acids having a central double-stranded region and hairpin regions at each end of the double-stranded region. Methods for the preparation and use of cyclic templates such as SMRTBELL® templates are described for example in U.S. Pat. No. 8,003,330, entitled “Error-free amplification of DNA for clonal sequencing”, and U.S. Patent Application Publication No. US2009/0280538, entitled “Methods and compositions for nucleic acid sample preparation”, the full disclosures of which are hereby incorporated by reference herein for all purposes.

### Kits

The present disclosure also provides applied embodiments of the methods and compositions disclosed herein.

For example, in certain embodiments, the present disclosure provides kits that are used for enriching for nucleic acids comprising a target sequence from a mixture of nucleic acids as described herein. A first exemplary kit provides the materials and methods for the attachment of strand-linking and non-strand-linking adapters to double stranded nucleic acids. The double-stranded nucleic acids can be from any desired sample or combination of samples. In some embodiments, reagents for the isolation of the double stranded nucleic acids (e.g., cell free DNA from a subject) are also present in the kit. As such, the kit will typically include those materials that are required to prepare a mixture of nucleic acids having adapters as outlined herein, e.g., in accordance with the various preparation processes outlined above. As will be appreciated, depending upon the nature of the adapter-attached nucleic acid construct and the method used, the kit contents can vary. For example, where one is employing a hairpin adapter and a linear adapter that are to be coupled to ends of double stranded nucleic acid segments, the kits will typically include such different adapters (e.g., with ends that are compatible with ends on the desired nucleic acids to be tagged, e.g., blunt and/or having a 3′ T overhang), along with appropriate ligation enzymes and protocols for attaching such adapters to the ends of double stranded nucleic acids, as well as any processing enzymes that may be desirable for treating the ends of the double stranded segments prior to ligation, e.g., phosphatases, exonucleases, and the like to provide blunt or 3′ A overhangs. In some cases, these kits may include enzyme systems for providing 5′ phosphate groups to the ends of fragments. The kits may further include reagents for performing nucleic acid synthesis reactions, including but not limited to synthesis primers specific for synthesis primer binding sites in one of the adapters, e.g., in the loop region of the hairpin adapter, a strand displacing polymerase and buffers/regents for hybridizing the synthesis primer and performing a nucleotide polymerase reaction. In some embodiments, the strand displacing polymerase is selected from a Φ29 DNA polymerase or modified version thereof, a homolog of a Φ29 DNA polymerase or modified version thereof, and combinations thereof. As the polymerase binding and nucleic acid synthesis steps may be performed under different reaction conditions, separate buffers/reagents can be provided for each. Alternatively, a single set of buffers/reagents for simultaneous polymerase binding and nucleic acid synthesis may be provided. In some cases, specific nucleotides are included in the reagents for nucleic acid synthesis, e.g., dUTP, which is used in certain methods described herein in conjunction with a USER enzyme mix to aid in the degradation of nascent nucleic acid strands (as detailed elsewhere herein). Kits can also include solid substrates that include a first member of a binding pair specific for the corresponding second member of the binding pair that is present as the retrieval region on the capture probe(s) specific for the target region(s) of interest. In some embodiments, kits also include one or more capture probes specific for one or more target regions of interest, where the one or more capture probes containing a retrieval region having the second member of the binding pair corresponding to the first member present on the solid substrate solid substrate. Binding members of a binding pair can include a nucleic acid sequence, biotin, avidin, streptavidin, digoxigenin, a protein, an antibody, or combinations thereof. In other embodiments, the capture probe is coupled to the solid substrate, either covalently or non-covalently (e.g., through a binding pair interaction). The one or more nucleases are selected from the group consisting of: an exonuclease that degrades RNA in an RNA/DNA heteroduplex; an exonuclease having 3′ to 5′ exonuclease activity on dsDNA; an exonuclease having 5′ to 3′ exonuclease activity on dsDNA; an exonuclease having 3′ to 5′ exonuclease activity on single stranded DNA; an exonuclease having 5′ to 3′ exonuclease activity on single stranded DNA; an uracil-specific excision reagent (USER); RNase H; Lambda exonuclease; exonuclease I; exonuclease III; and any combination thereof.

In addition, kits may include reagents for removing undesired nucleic acids in the sample during or after adapter ligation (but before enrichment of nucleic acids having a target region of interest), including exonucleases, nucleic acid purification columns or beads, size-selection columns or spin tubes, affinity/capture reagents (e.g., biotin, avidin, capture probes specific for universal capture sites in adapters (not used for target specific enrichment), etc.). Further, kits may include reagents for generating the initial nucleic acid fragments to be tagged, including nucleic acid isolation reagents, fragmentation reagents (e.g., fragmentation columns, restriction enzymes, etc.).

A second exemplary kit provides materials and methods not just for the enrichment of nucleic acids from a mixture having one or more region of interest, but also for the sequencing of such enriched nucleic acids. Thus, in addition to the materials and methods set forth above, such kits may additionally include reagents used in such sequencing processes, such as primer sequences for initiating the sequence process, polymerase enzymes, and substrates that provide for optical confinement of nucleic acid synthesis complexes. In certain aspects, such substrates will typically include one or more arrays of zero mode waveguides (ZMW). Such waveguide arrays may further include surface treatments that provide for enhanced localization of synthesis complexes within the illumination volumes of such zero mode waveguides, e.g., as described in Published International Patent Application No. WO 2007/123763, incorporated herein by reference in its entirety for all purposes. Additionally, such kits may optionally include nucleotide compositions for use in sequencing applications, including, for example labeled nucleotides that include fluorescent or otherwise detectable labeling groups coupled to the phosphate groups in a nucleoside polyphosphate construct at a phosphate group other than the alpha phosphate. A variety of other types of labeled and unlabeled nucleotides may be optionally includes within the kits and are generally known in the art.

### Specific Embodiments in the Figures

The specific embodiments described below are meant to illustrate aspects of the present disclosure but are not meant to be limiting.

FIG. 1 illustrates an embodiment of the invention for the enrichment of nucleic acids that contain a target sequence of interest from a mixture.

In FIG. 1, a sample of double stranded DNA fragments 100 is provided. The double stranded DNA fragments have ligation-competent ends that prevent concatemer formation, in this case by including a 5′ phosphate group (“p” on the fragment ends in FIG. 1) and a 3′ dA overhang (“A” on the fragment ends in FIG. 1), e.g., by end repair of double stranded DNA fragments in the presence of Taq DNA polymerase and T4 polynucleotide kinase as known in the art.

In step (1), a 1:1 mixture of a hairpin adapter 102 (a strand-linking adapter) and a linear adapter 104 (a non-strand-linking adapter) that have ligation-competent ends compatible with the ends of the DNA fragments (having a 3′ dT overhang and 5′ phosphate) are combined with the DNA fragments and ligase and placed under reaction conditions that allow ligation of the adapters to the DNA fragments. The adapter mixture is generally provided in molar excess to the DNA fragments in sample 100 to drive the reaction to completion. In FIG. 1, the linear adapter 104 is protected from exonuclease degradation at one end by the inclusion of exonuclease-resistant nucleotides 106 (in this case, nucleotides with thiophosphate linkages). The resulting adapter-ligated DNA fragment population 108 (“tagged” DNA) includes approximately 50% asymmetrically-tagged DNA fragments 110 (DNA inserts having a hairpin adapter at a first end and a linear adapter at the second end) and 50% symmetrically tagged DNA fragments 112 (DNA inserts having either hairpin adapters or linear adapters at both ends).

As indicated elsewhere, adapters used to tag DNA fragments can include any convenient functional regions, domains or sequences that find use in downstream steps, processes or analyses. For example, if the resultant DNA fragments are to be employed in a multiplex analytical process, barcode sequences can be preset in one or more of the adapters used (including the second hairpin adapter described below). Adapters can thus include, for example, one or more of: restriction enzyme sites, sequencing/synthesis primer binding sites, barcode sequences, universal capture probe binding sites, site or structure designed for enzyme binding (e.g., helicase binding as employed in nanopore sequencing), etc.

In step (2), a capture probe 114 is combined with the tagged DNA fragments. In this example, the capture probe 114 has a target sequence binding region 116 that includes an RNA sequence complementary to all or a portion of one or more target sequence of interest and a biotin moiety that serves as a retrieval region 118. The sample is placed under denaturation conditions and then conditions that allow annealing of the target sequence binding region 116 to its cognate complementary sequence (these conditions are sometimes referred to as renaturation conditions). The denaturation/renaturation is achieved in this example by heating and cooling the sample as is known in the art. After renaturation, the capture probe 114 will be bound to DNA inserts in the mixture of tagged DNA fragments that include the target sequence of interest via the target sequence binding region 116 to form complex 120. (It is noted that only DNA fragments having the target sequence of interest are shown for simplicity; there are many DNA fragments that do not include the target sequence of interest and are thus do not have a hybridized capture probe.)

In step (3), the renatured sample is contacted to streptavidin beads (SA-beads) 122 under biotin/streptavidin binding conditions which results in the binding of complex 120 (i.e., capture probes bound to target sequences in DNA inserts) to the SA-beads through the interaction of the biotin moieties 118 on the capture probe with the SA moieties on the SA beads. Non-bound material, including tagged DNA fragments without a bound capture probe (and thus not having the target sequence of interest), is washed away.

In step (4), bound tagged DNA fragments are eluted from the streptavidin beads by treatment with RNase H to degrade the RNA-based target binding region 116 of the capture probe that is bound to the target sequence in the DNA insert (RNase H degrades RNA in RNA/DNA duplexes). The sample eluted from the beads is enriched for DNA fragments 124 that have the target sequence of interest.

In steps (5) and (6) of FIG. 1, the DNA fragments 124 are renatured (which can happen during the elution process itself, and thus may not need a separate step) and cleaved with a restriction enzyme that recognizes a cleavage site 126 in the linear adapter 104 to generate DNA fragments 128 each having ligation site 130. The restriction enzyme cleavage site 126 can be selected from any site that is predicted to not be present in DNA inserts having the target sequence of interest, e.g., a rare Type IIS restriction enzyme site (e.g., BsaI as shown in FIG. 1). In step (7), a second hairpin adapter 132 having end 134 that is compatible with ligation site 130 is combined with the DNA fragments with a ligase to produce double-hairpin tagged DNA fragments 136. This enriched sample containing double hairpin tagged fragments is then treated with exonuclease to degrade any excess hairpin adapters in step (8). As the sequence of the second hairpin 132 adapter may be the same or different from the sequence of the first hairpin adapter 102, tagged DNA fragment 136 may be symmetric or asymmetric, depending on the desires of the user. This sample of target sequence enriched DNA fragments can be purified to remove contaminants and/or concentrated (if either is needed) and employed for any desired downstream process or analysis, e.g., SMRT® Sequencing.

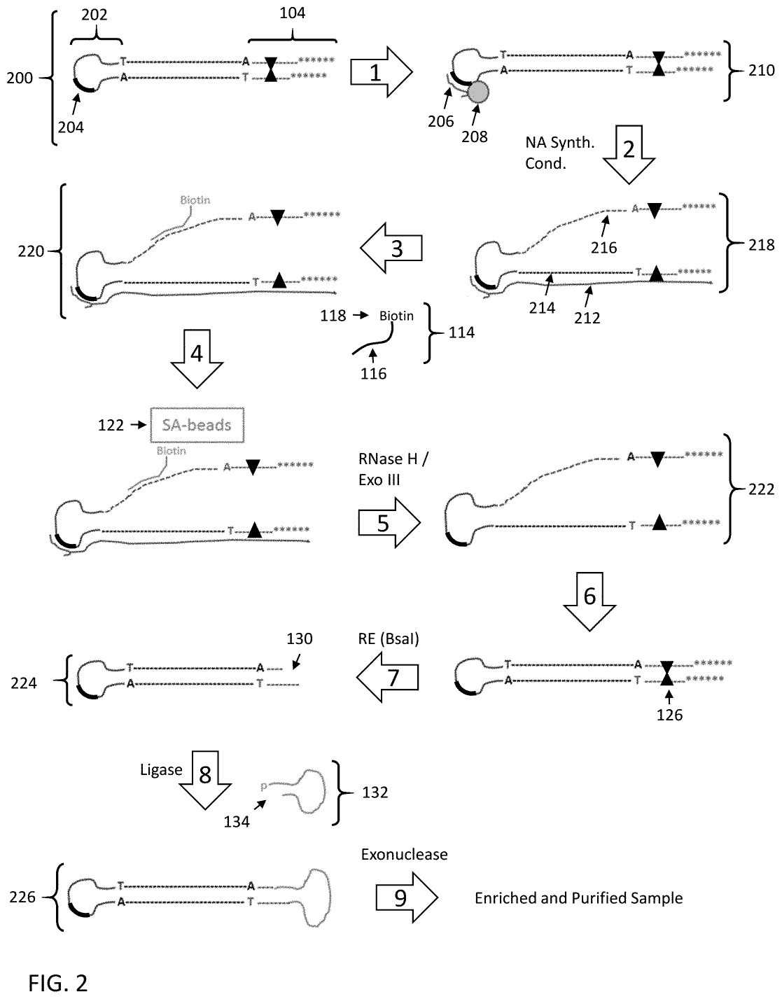

FIG. 2 illustrates another embodiment of the invention for the enrichment of nucleic acids that contain a target sequence of interest from a mixture.

Unlike FIG. 1, FIG. 2 does not show the step of generating of the asymmetrically-tagged DNA fragment population 200. The process is generally the same, except that in FIG. 2 the hairpin adapter 202 includes a nucleic acid synthesis primer binding site 204; the linear adapter 104 has the same structure as shown in FIG. 1.

In step (1) a nucleic acid synthesis primer 206 and a polymerase enzyme 208 are added to the adapter-ligated DNA fragment population 200 under appropriate reaction conditions to promote the formation of polymerase-nucleic acid complexes 210, in which primer 206 anneals to primer binding site 204 forming a site at which polymerase 208 can bind. The reaction conditions include the appropriate salts, metals, buffers, etc., during complex formation. As is well known in the art, the polymerase enzyme 208 is able to identify and bind to the appropriate location at the 3′ end of synthesis primer 206 poised for nucleic acid synthesis. In some cases, it is desirable to add an excess of polymerase enzyme in step (1) to ensure a high yield of nucleic acid synthesis on the tagged DNA fragments. For example, in some cases, molar ratios of 10:1 to 50:1 of polymerase enzyme to nucleic acid are used.

In step (2), nucleic acid polymerization is initiated, resulting in the formation of a nascent strand 212 extended from the primer 206 on strand 214 of the DNA fragment (the template strand). In order to initiate polymerization, all of the required components, including all necessary nucleotides are added to the solution containing the complex 210. The polymerase enzyme that is used in this step has strand displacement activity, e.g., a phi29-type DNA polymerase, which results in displacement of the second strand 216 that is complementary to the template strand 214, forming open complex 218. Because this process produces a displaced single stranded region (second strand 216), it effectively “denatures” the DNA fragment and readies the second strand for capture probe binding (when the target sequence is present in the second strand 216). It is noted that in some embodiments, steps (1) and (2) are performed in a single step.

In step (3) a capture probe 114 specific for the target sequence of interest is combined with the polymerase-denatured DNA fragment 218 under nucleic acid hybridization conditions. As with FIG. 1, the capture probe 114 has a target sequence binding region 116 that includes an RNA sequence complementary to all or a portion of one or more target sequence of interest and a biotin moiety that serves as a retrieval region 118. The capture probe hybridizes to its cognate target sequence in the second strand 216 that was exposed by the action of the polymerase enzyme through the target sequence binding region 116 to form complex 220.

In step (4) the capture probe hybridized sample is contacted to streptavidin beads 122 under biotin/streptavidin binding conditions which results in complex 220 (i.e., capture probes bound to cognate target sequences in DNA inserts) being bound to the beads. Non-bound material, including tagged DNA fragments without a bound capture probe (and thus not having the target sequence of interest), inactive polymerase-nucleic acid complexes, and excess uncomplexed polymerase enzyme, is washed away. In some cases, this step may include adding a polymerase trap prior to washing. A polymerase trap is used to bind excess free polymerase within the reaction in order to more effectively remove it from the desired polymerase-nucleic acid complex attached to the bead through the capture probe. One useful polymerase trap is heparin, to which polymerases are known to bind. Nucleic acids such as DNA can also be used as polymerase traps to assist in removal of the excess polymerase. In some cases, single stranded DNA such as circular single stranded DNA can be used.

In step (5), bound tagged DNA fragments are eluted from the streptavidin beads by treating the streptavidin beads with RNase H to degrade the RNA-based target binding region of the capture probe 116. The sample eluted from the beads is enriched for DNA fragments that have a displaced strand 216 containing the target sequence of interest and a double stranded region containing hybridized template strand 214 and nascent strand 212.

Step (5) also includes treating the eluted DNA fragments with a 3′ exonuclease, e.g., exonuclease III or Lambda exonuclease, to degrade nascent strand 212 (as noted above and shown in FIG. 1, linear adapter 104 is protected from exonuclease degradation by the exonuclease-resistant nucleotides 106) to produce a sample enriched for DNA fragments 222 that have the target sequence of interest.

In steps (6) and (7) of FIG. 2, which are analogous to steps (5) and (6) in FIG. 1, the enriched DNA fragments 222 are renatured and cleaved with a restriction enzyme that recognizes a cleavage site 126 in the linear adapter 104 to generate DNA fragments 224 each having ligation site 130. The restriction enzyme cleavage site 126 can be selected from any site that is predicted to not be present in DNA inserts having the target sequence, e.g., a rare Type IIS restriction enzyme site (e.g., BsaI as shown in FIG. 2). In step (8), analogous to step (7) in FIG. 1, a second hairpin adapter 132 having end 134 that is compatible with ligation site 130 is combined with the DNA fragments with a ligase to produce double-hairpin tagged DNA fragments 226. Second hairpin adapter 132 can have the same sequence as adapter 202 or at least one sequence difference from adapter 202 (as noted in FIG. 1). As such, double-hairpin tagged DNA fragments 226 can be symmetrically or asymmetrically tagged. This enriched sample containing double hairpin tagged fragments is then treated with exonuclease to degrade any excess hairpin adapters in step (9). This sample of target sequence-enriched DNA fragments can be purified to remove contaminants and/or concentrated (if either is needed) and employed for any desired downstream process or analysis, e.g., SMRT® Sequencing.

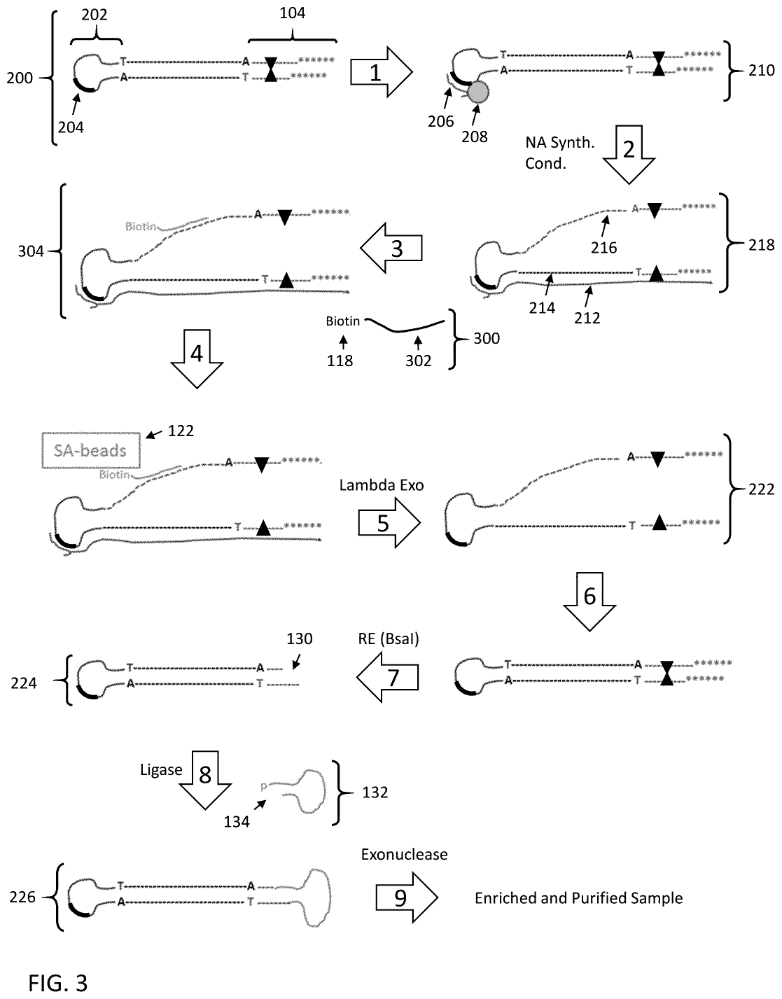

FIG. 3 illustrates an embodiment similar to FIG. 2, and thus not all steps in this figure are described in full below.

In contrast to FIG. 2, however, in step (3) of FIG. 3 a capture probe 300 is used in which the target sequence binding region 302 includes a DNA sequence (rather than an RNA sequence) that is complementary to all or a portion of one or more target sequences of interest. Hybridization of the capture probe 300 to a cognate target region in the single strand 216 forms complex 304. The capture probe 300 still includes a biotin moiety 118 that serves as a retrieval region, in this case present at the 3′ end of the capture probe. In step (4) the capture probe hybridized sample is contacted to streptavidin beads 122 under biotin/streptavidin binding conditions which results in complex 304 (i.e., capture probes bound to cognate target sequences in DNA inserts) being bound to the beads 122. Non-bound material, including tagged DNA fragments without a bound capture probe (and thus not having the target sequence of interest), inactive polymerase-nucleic acid complexes, and excess uncomplexed polymerase enzyme, is washed away. Because DNA is used as the target sequence binding region 302, a 5′-3′ exonuclease specific for dsDNA (e.g., Lambda Exonuclease) is used to degrade the hybridized capture probe annealed to the target sequence in the DNA fragments (step (5)) to elute the DNA fragments from the streptavidin beads after capture step (4). As such, no RNase H treatment is needed (as was done in FIGS. 1 and 2). The exonuclease used in step (5) will also remove nascent strand 212 from DNA complex 304 [generated in previous step (2) to displace strand 216 (as in FIG. 2)], and thus obviates the need for an additional exonuclease to achieve this outcome. After exonuclease treatment, the eluted DNA 222 is renatured and processed as described in FIG. 2 above (shown as steps (6) to (9) in FIG. 3).

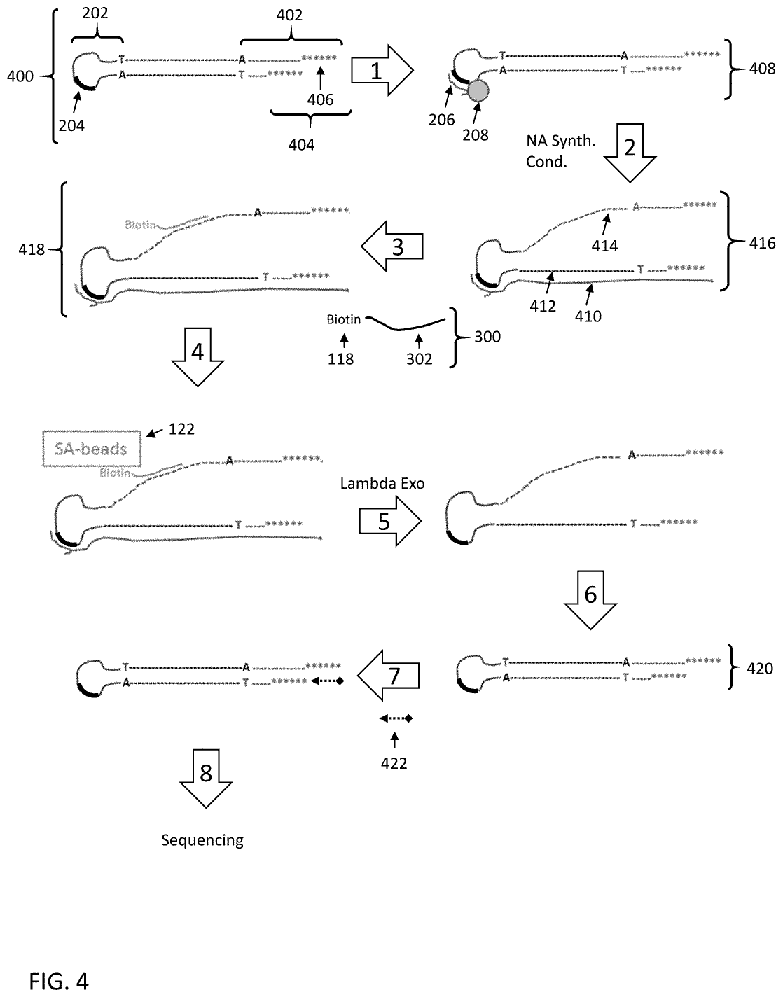

FIG. 4 illustrates an embodiment similar to FIGS. 2 and 3, and thus not all steps in this figure are described in full below.

In contrast to FIGS. 2 and 3, however, the linear adapter 402 in FIG. 4 that is ligated to asymmetrically-tagged DNA fragment population 400 has an exonuclease resistant end 404 (noted by *s) that includes a 3′ overhang region 406. All or a portion of this single stranded 3′ overhang 406 can serve as a sequencing primer binding site. Steps (1) through (6) are performed in the same manner as in FIG. 3 to produce a sample enriched for DNA fragments 420 having the target sequence of interest. In brief, nucleic acid synthesis in step (2) produces denatured complex 416 having template strand 412, nascent strand 410, and single stranded region 414. In step (3) capture probe 300 is annealed to its cognate target sequence in displaced strand 414 to form complex 418. In step (4), complex 418 is contacted to SA-beads 122 under appropriate biotin/streptavidin binding conditions. Captured complexes are eluted from SA-beads 122 using a 3′-5′ exonuclease (which also removes the nascent strand) and renatured in steps (5) and (6) to produce enriched DNA fragments 420. At step (7), rather than cleavage with a restriction enzyme and ligation of a second hairpin adapter as in the previous embodiment, the enriched DNA fragments 420 are primed with a sequencing primer 422 specific for the sequencing primer binding site present in 3′ overhang region 406 and then subjected to a sequencing-by-synthesis reaction, e.g., a SMRT® Sequencing reaction. This process is particularly useful when sequencing long inserts, e.g., inserts that are about 5 kilobases (kb) or more, 10 kb or more, 20 kb or more, or 50 kb or more in length.

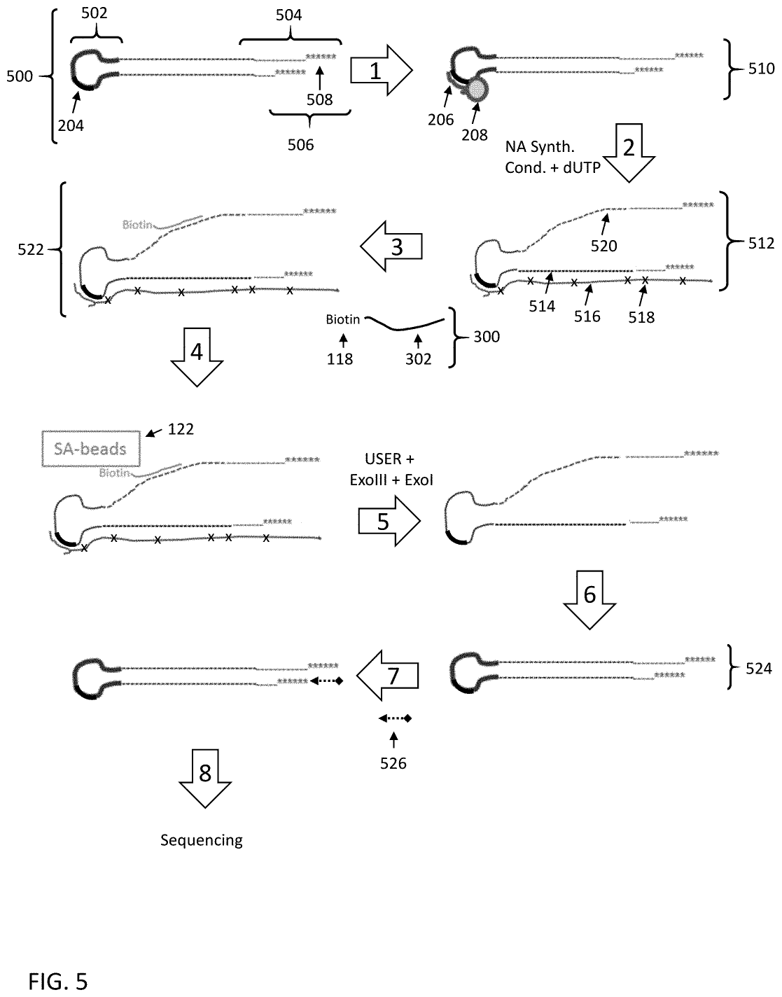

FIG. 5 illustrates an embodiment similar to FIG. 4, but with certain differences as detailed below.

In FIG. 5, the hairpin adapter 502 and the linear adapter 504 are ligated to DNA fragments to form asymmetrically-tagged population 500 using blunt-end ligation (this step is not shown, but it is indicated by the lack of the A and T residues in the asymmetrically-tagged DNA fragments). It is noted that blunt-end adapter ligation can be used to form tagged DNA fragments in other embodiments (e.g., in FIGS. 1 to 4) and is not meant to be limited to the embodiment shown in this figure. Linear adapter 504 has an exonuclease resistant end 506 (noted by *s) that includes a 3′ overhang region 508. All or a portion of this single stranded 3′ overhang 508 can serve as a sequencing primer binding site.

In step (1) a nucleic acid synthesis primer 206 and a polymerase enzyme 208 are added to the adapter-ligated DNA fragment population 200 under appropriate reaction conditions to promote the formation of polymerase-nucleic acid complexes 510, in which primer 206 anneals to primer binding site 204 forming a site at which polymerase 208 can bind (similar to previous embodiments). The reaction conditions include the appropriate salts, metals, buffers, etc., during complex formation.

In step (2), nucleic acid polymerization is initiated as described in previous embodiments, except that in this embodiment dUTP is included in the nucleotide mix. This results in the formation of a nascent strand 516 extended from the primer 206 on template strand 514 of the DNA fragment that includes dU moieties 518 in nascent strand 516 (indicated by “x” in the nascent strand). Because the polymerase enzyme that is used has strand displacement activity (as noted above), the strand 520 is displaced from template strand 514 forming open complex 512.

In step (3) capture probe 300 is annealed to its cognate target sequence, via target sequence binding region 302, in displaced strand 520 to form complex 522. In step (4), complex 522 is contacted to SA-beads 122 under appropriate biotin/streptavidin binding conditions to allow binding of biotin moiety 118 to streptavidin.

In contrast to previous embodiments, captured complexes are eluted from SA-beads 122 step (5) in FIG. 5 using a combination of enzymes that includes: an enzyme with dsDNA 3′-5′ exonuclease activity (e.g., ExoIII); an enzyme that has ssDNA 3′ to 5′ and/or 5′ to 3′ exonuclease activity (e.g., ExoI); and a USER enzyme mix (Uracil-Specific Excision Reagent). The USER enzyme mix includes a Uracil DNA glycosylase (UDG) and an Endonuclease VIII enzyme (EndoVIII). The UDG catalyzes the excision of the uracil bases in nascent strand 516 forming abasic (apyrimidinic) sites while leaving the phosphodiester backbone intact. The lyase activity of EndoVIII breaks the phosphodiester backbone at the 3′ and 5′ sides of the abasic sites so that base-free deoxyribose is released, thus exposing 3′ OH recognition sites for ExoIII (or similar) enzyme in nascent strand 516. This combination of enzymes efficiently degrades both nascent strand 516 and the capture oligo 300.

The enriched DNA fragments 524 produced in step 6 can be used in any downstream process or analysis. In some embodiments, and as shown in step (7), the enriched DNA fragments 524 are primed with a sequencing primer 526 specific for the sequencing primer binding site present in 3′ overhang region 508 and then subjected to a sequencing-by-synthesis reaction (as in step (8)), e.g., a SMRT® Sequencing reaction. As noted in the previous embodiments, this process is particularly useful when sequencing long inserts, e.g., inserts that are about 5 kilobases (kb) or more, 10 kb or more, 20 kb or more, or 50 kb or more in length.

FIG. 6 illustrates an alternative embodiment for the enrichment of nucleic acids that contain a target sequence of interest from a mixture.

In FIG. 6, a population of nucleic acids 600 are symmetrically tagged with a hairpin adapter 602 (in this case at compatible T/A overhangs as indicated by the T and A nucleotides shown) that includes a primer binding site 204. The population is shown with a nucleic acid synthesis primer 206 hybridized to site 204 with a polymerase enzyme 208 complexed thereto. As detailed above, the conditions in which this nucleic acid/primer/polymerase complex is formed includes the appropriate salts, metals, buffers, etc.

In step (1), nucleic acid polymerization is initiated in the presence of dUTP to form complex 610. This results in the formation of a nascent strands 608 extended from the primers 206 that includes dU moieties. (It is noted that while two nascent strands are show in FIG. 6, there will likely be nucleic acids that have only one productive primer/polymerase complex, and thus will generate only one nascent strand. These single nascent strand extension products will also be selected in the steps shown in FIG. 6 and described below.) Because the polymerase enzyme that is used has strand displacement activity (as noted above), nascent strands 608 can produce multiple copies of the nucleic acid insert and adapters, or concatemers. While the nascent strand concatemers 608 in complex 610 are shown as re-forming their insert/hairpin secondary structure, this is merely for convenience. No limitation with respect to the secondary structure of the nascent strand concatemers 608 is intended.

In step (2) capture probe 300 is contacted under appropriate nucleic acid hybridization conditions to allow annealing of the target sequence binding region 302 to its cognate target sequence in nascent strands 608 without disrupting the association of the nascent strands with the template strand (i.e., the original nucleic acid 600) to form capture primer hybridized complex 612.

In step (3), complex 612 is contacted to SA-beads 122 under appropriate biotin/streptavidin binding conditions to allow binding of biotin moieties 118 to streptavidin. Non-binding material, i.e., material that does not include capture probe 300 is removed (e.g., washed away).

Captured complexes are eluted from SA-beads 122 in step (4) in FIG. 6 using a combination of enzymes that includes: an enzyme with dsDNA 3′-5′ exonuclease activity (e.g., ExoIII); an enzyme that has ssDNA 3′ to 5′ and/or 5′ to 3′ exonuclease activity (e.g., ExoI); and a USER enzyme mix (Uracil-Specific Excision Reagent). The USER enzyme mix includes a Uracil DNA glycosylase (UDG) and an Endonuclease VIII enzyme (EndoVIII). The UDG catalyzes the excision of the uracil bases in nascent strand 608 forming abasic (apyrimidinic) sites while leaving the phosphodiester backbone intact. The lyase activity of EndoVIII breaks the phosphodiester backbone at the 3′ and 5′ sides of the abasic sites so that base-free deoxyribose is released, thus exposing 3′OH recognition sites for ExoIII (or similar) enzyme in nascent strand 608. This combination of enzymes efficiently degrades both nascent strand 608 and the capture oligo 300.

The enriched DNA fragments 614 produced in step (4) can be used in any downstream process or analysis. In some embodiments, and as shown in step (5), the enriched DNA fragments 614 are primed with a sequencing primer 206 specific for the primer binding site 204 present in hairpin adapter 602 and then subjected to a sequencing-by-synthesis reaction (as in step (6)), e.g., a SMRT® Sequencing reaction. It is noted that a different primer can be employed if desired (e.g., one that recognizes a different primer binding site in the hairpin adapter 602).

FIG. 7 illustrates an alternative embodiment for the enrichment of nucleic acids that contain a target sequence of interest from a mixture.

Steps (1) to (3) in FIG. 7 are similar to FIG. 6 except that the nucleic acid synthesis reaction performed on the population of nucleic acids 600 in step (1) does not include dUTP. Thus, the nascent strands 702 in complexes 704 do not include dU moieties. (It is again noted that while two nascent strands are show in FIG. 7, there will likely be nucleic acids that have only one productive primer/polymerase complex, and thus will generate only one nascent strand.) Because the polymerase enzyme that is used has strand displacement activity (as noted above), nascent strands 702 can produce multiple copies of the nucleic acid insert and adapters, or concatemers. While the nascent strand concatemers 702 in complex 704 are shown as re-forming their insert/hairpin secondary structure, this is merely for convenience. No limitation with respect to the secondary structure of the nascent strand concatemers 702 is intended.

Steps (2) and (3) in FIG. 7 (capture probe 300 hybridization and SA-bead 122 binding) is carried out as described in FIG. 6.

Captured complexes are eluted from SA-beads 122 in step (4) in FIG. 7 using an enzyme with dsDNA 3′-5′ exonuclease activity (e.g., ExoIII) which will digest the nascent strand hybridized to the original nucleic acid 600. The exonuclease enzyme will not degrade DNA at single stranded locations, and thus will stop once it has reached the location at which the nascent strand 702 is no longer hybridized to the original template nucleic acid 600 (i.e., where the nascent strand has been separated from the template by the action of the strand-displacing polymerase). This treatment results in the production of enriched species 708 (representing the original nucleic acid having the region of interest) and 710 (representing nascent strands having newly-synthesized regions of interest).

Enriched DNA fragments 708 and 710 produced in step (4) can be used in any downstream process or analysis. In some embodiments, and as shown in step (5), enriched DNA fragments 708 are primed with a sequencing primer 206 specific for the primer binding site 204 present in hairpin adapter 602 and then subjected to a sequencing-by-synthesis reaction (as in step (6)), e.g., a SMRT® Sequencing reaction. It is noted that a different primer can be employed if desired (e.g., one that recognizes a different primer binding site in the hairpin adapter 602). In additional embodiments, enriched nascent strands 710 are primed with a different sequencing primer 712 and subjected to a sequencing-by-synthesis reaction (as in step (8)), e.g., a SMRT® Sequencing reaction. The design of sequencing primer 712 needs to take into account that the single-stranded hairpin regions in nascent strands 708 are complementary to the single-stranded regions in hairpin adapters 602. In some embodiment, both enriched fragments 708 and 710 are sequenced.

In some embodiments, certain enriched nucleic acids are sequenced in a nanopore sequencing method, e.g., enriched nucleic acids having a strand-linking adapter at one end and a linear adapter at the second end. In many of these embodiments, the linear adapter includes features and/or moieties that facilitate nanopore loading and sequencing (e.g., nanopore adapters with membrane- or nanopore-targeting moieties, loading moieties, strand separating moieties (e.g., helicases, polymerases), and the like). For example, in FIG. 1, adapter 104 could be designed to be a nanopore adapter such that enriched nucleic acids 124 can be sequenced without further adapter ligation. Alternatively, adapter 132 in step 7 could be a nanopore adapter. Similar modifications could be made to the methods shown in FIGS. 2, 3, 4, and 5 to allow for enriched nucleic acids to be sequenced by nanopore sequencing. In FIG. 7, concatemers 710 can be used as templates for nanopore sequencing, e.g., either directly or after attaching nanopore adapters to an end. In some embodiments, enriched nucleic acids 708 are sequenced in a SMRT sequencing process and enriched nucleic acids 710 are sequenced in a nanopore sequencing process, with the resulting sequencing data from both processes analyzed as desired by the user.

It will be readily apparent to one of ordinary skill in the relevant arts that other suitable modifications and adaptations to the methods and compositions described herein can be made without departing from the scope of the invention or any embodiment thereof. Having now described the present invention in detail, the same will be more clearly understood by reference to the following Examples, which are included herewith for purposes of illustration only and are not intended to be limiting of the invention.

### EXAMPLES

### Example 1

The following example employs the strategy as set forth in FIG. 4 to enrich specific nucleic acid fragments from a HindIII digested Lambda DNA sample with asymmetric adapters (a hairpin adapter and a linear adapter).

Lambda/HindIII Library Construction:

Lambda DNA was digested to completion with HindIII, end repaired and treated to generate ends having a 3′-A overhang. Hairpin and linear adapters having compatible 3′-T overhangs mixed at a 1:1 molar ratio were added to the digested Lambda DNA fragments under DNA ligation conditions. The hairpin adapter includes a synthesis primer binding site in the single stranded loop region. The linear adapters included a 3′ overhang region on the end opposite the 3′-T ligation site that included a sequencing primer binding site. In addition, the end opposite the 3′-T ligation site included 5′ and 3′ terminal phosphorothioate nucleotides to protect them from exonuclease digestion once the 3′-T ligation site was ligated to a compatible end of a DNA fragment. The ligation reaction was treated with exonucleases to degrade nucleic acids with free, unprotected 5′ and/or 3′ ends (DNA fragments with at least one unligated end and free adapters). After exonuclease treatment, the adaptor ligated templates were purified using AMPure beads according to manufacturer's instructions. The resultant adapter-ligated DNA fragments were estimated to be ˜50% asymmetrically tagged (having a hairpin adapter at one end and a linear adapter at the opposite end) and ˜50% symmetrically tagged (having the same adapter at both ends).

Target Enrichment and Sequencing:

Two different DNA fragments from the Lambda/HindIII library generated above were selected for enrichment: the 6.6 kb fragment (nucleotides 37,584-44,141) and the 9.4 kb fragment (nucleotides 27,479-36,895). The nucleotide sequences of the 3′ biotinylated capture probes are listed in Table 1 and were purchased from IDT (Integrated DNA Technologies, Inc.; Skokie, Ill.). (The 3′ biotin tag is not shown in table 1.) The melting temperature (Tₘ) in ° Centigrade for each probe is listed. The first two probes are specific for the 6.6 kb fragment and the last four are specific for the 9.4 kb fragment. The numbers listed in the name represent the location in the Lambda DNA that each probe sequence is based on while the F and R in the name indicate which strand the probe sequence is based on (the “forward” or “reverse” strand). Both strands of each of the target fragments are targeted for enrichment to allow enrichment regardless of the orientation of the hairpin adapter (closed adapter) and the linear adapter (open adapter).

TABLE 1
Enrichment Probes

| Name | Sequence | Tₘ (° C.) |
|---|---|---|
| Kda1F_B50_40811-40860: | 5′ CTC TCG TCA GGT TGA ATG GCA TGG TCG CTG | 72.1 |
| | GCT GGA TGC AGA AAG CTG GA-3′ | |
| | [SEQ ID NO: 1] | |
| Lda1R_B57_40929-40873: | 5′ CCA CAA AGC CAT TCC CGG CAA GGT TAG GAA | 71.7 |
| | CAA CAT CCT GCT GCT TTA ATG CTG CGG 3′ | |
| | [SEQ ID NO: 2] | |
| Lda2F_B50_35551-35600: | 5′ CAC CTT CAT GGT GGT CAG TGC GTC CTG CTG | 71.9 |
| | ATG TGC TCA GTA TCA CCG CC 3′ | |
| | [SEQ ID NO: 3] | |
| Lda2R_B51_35819-35769: | 5′ CCT CAG CGC CGG GTT TTC TTT GCC TCA CGA | 72.3 |
| | TCG CCC CCA AAA CAC ATA ACC ′3 | |
| | [SEQ ID NO: 4] | |
| Lda3F_B53_31928-31980: | 5′ GCG GTG ATG ACG CCG AGC CGT AAT TTG TGC | 73.6 |
| | CAC GCA TCA TCC CCC TGT TCG AC 3′ | |
| | [SEQ ID NO: 5] | |
| Lda3R_B53_32069-32017: | 5′ GGA TTC CTG AAA CAG AAA GCC GCA GAG CAG | 72.2 |
| | AAG GTG GCA GCA TGA CAC CGG AC 3′ | |
| | [SEQ ID NO: 6] | |

To separate the strands of the hairpin-ligated DNA fragments in the Lambda/HindIII DNA library, a nucleic acid synthesis reaction was initiated (using a cognate synthesis primer and a wild-type phi-29 DNA polymerase) from the nucleic acid synthesis primer binding site present in the loop region of the hairpin adapter. The reaction was allowed to proceed for 30 minutes. The reaction was stopped by heating to 65° C. for 10 minutes. Primer-extended complexes were exposed to the 6 biotin-probes listed in Table 1 for 10 minutes at 65 C and 18 hours at 30° C. and then to streptavidin (SA)-beads according to the manufacturer's (IDT) protocol. After removal of the supernatant and washing, captured complexes were released and isolated from the SA-beads by incubation with T7 Exonuclease, which digests the IDT probes and the nascent DNA strands synthesized to separate the strands. The isolated DNA fragments were sequenced in a Single Molecule, Real-Time (SMRT®) Sequencing reaction (Pacific Biosciences of California, Inc.) using a sequencing primer specific for the sequencing primer binding site present in the 3′ overhang region of the linear adapter.

Results:

Table 2 shows the percent of mapped reads derived from the indicated Lambda/HindIII fragments (6.6 kb and 9.4 kb) from samples that were and were not subjected to enrichment as described above.

TABLE 2

% of the Mapped Reads* Derived from Fragment

| Fragment | No Enrichment | Enriched |
|---|---|---|
| 6.6 kb | 12.2 | 28.5 |
| 9.4 kb | 20.9 | 43.5 |

*Mapped reads are reads that are obtained during a 20 second filter that map to a single specific contiguous region in Lambda genomic DNA.

As seen in Table 2, the enrichment method increased the percent of mapped reads derived from the targeted fragments (6.6 kb and 9.4 kb) as compared to the Lambda/HindIII library prior to enrichment. more reads are mapped to the targeted 6.6 kb and 9.4 kb fragments from Lambda/Hind III library.

### Example 2

The following example employs the strategy as set forth in FIG. 7 to enrich specific nucleic acid fragments from a HindIII digested Lambda DNA sample with symmetric hairpin adapters (a hairpin adapter at both ends). In this example, the nascent DNA is sequenced rather than the original Lambda/HindIII template (similar to steps 7 and 8 of FIG. 7).

Lambda/HindIII SMRTBELL® Library Construction (symmetric hairpin adapter-ligated DNA fragments):

Lambda DNA was digested to completion with HindIII, end repaired, and treated to generate ends having a 3′-A overhang. Hairpin adapters having compatible 3′-T overhangs were added to the digested Lambda DNA fragments under DNA ligation conditions. The hairpin adapter includes a synthesis primer binding site in the single stranded loop region. The ligation reaction was treated with exonucleases to degrade nucleic acids with free 5′ and/or 3′ ends (Lambda DNA with at least one unligated end and free hairpin adapters). After exonuclease treatment, the library (Lambda/HindIII SMRTBELL® library) was purified using AMPure beads according to manufacturer's instructions.

A nucleic acid synthesis reaction was initiated (using a cognate synthesis primer and a phi-29 DNA polymerase) from the nucleic acid synthesis primer binding site present in the loop region of the hairpin adapter. The reaction was allowed to proceed for a time sufficient to enter rolling circle replication on the template (60 minutes). The reaction was stopped by heating to 65° C. for 10 minutes. Primer-extended complexes were exposed to the 6 biotin-probes listed in Table 1 for 10 minutes at 65° C. and 18 hours at 30° C. and then to streptavidin (SA)-beads according to the manufacturer's (IDT) protocol. After removal of the supernatant and washing, captured complexes were released and isolated from the SA-beads by incubation with Exonuclease III, which digests the IDT probes and the nascent DNA strands hybridized to the original Lambda/HindIII template. The isolated DNA fragments were sequenced in a Single Molecule, Real-Time (SMRT®) Sequencing reaction (Pacific Biosciences of California, Inc.) using a sequencing primer specific for the complement of the nucleic acid synthesis primer binding site present in the in the single stranded loop region of the hairpin adapter. This sequencing primer will hybridize to the nascent DNA strands, and not the original Lambda/HindIII template, and thus return sequence information for the nascent strand.

Results:

Table 3 shows the percent of mapped reads derived from the indicated Lambda/HindIII fragments (6.6 kb and 9.4 kb) from samples that were and were not subjected to enrichment as described above. The “no enrichment” sample was subjected to the initial nucleic acid synthesis step, and thus the sequencing information is also from the nascent strand and not the original template DNA.

TABLE 3

% of the Mapped Reads* Derived from Fragment

| Fragment | No Enrichment | Enriched |
|---|---|---|
| 6.6 kb | 12.9 | 24.0 |
| 9.4 kb | 15.2 | 34.8 |

*Mapped reads are reads that are obtained during a 20 second filter that map to a single specific contiguous region in Lambda genomic DNA.

As seen in Table 3, the enrichment method increased the percent of mapped reads derived from the targeted fragments (6.6 kb and 9.4 kb) as compared to the Lambda/HindIII library prior to enrichment. This result verifies that the enrichment process.

### Example 3

The following example employs the strategy as set forth in FIG. 7 to enrich specific nucleic acid fragments from a human genomic DNA library. The capture probes employed in this example target Alzheimer's Disease-related loci present on multiple different human chromosomes.

Human Genomic DNA Library Construction:

Human genomic DNA was fragmented by shearing and size-selected for fragments of approximately 6 kb in length using Covaris g-Tube. The size-selected DNA fragments were DNA-repaired and end repaired and treated to generate blunt ends. Blunt end hairpin and linear adapters were mixed at a 1:1 molar ratio and added to 4 μg of the size-selected blunt-end DNA fragments under DNA ligation conditions. The hairpin adapter included a synthesis primer binding site in the single stranded loop region. The linear adapter included a 3′ overhang region on the end opposite the blunt ligation site that included a sequencing primer binding site. In addition, the end opposite the blunt ligation site included 5′ and 3′ terminal phosphorothioate nucleotides to protect them from exonuclease digestion once ligated to a compatible end of a DNA fragment. The ligation reaction was treated with exonucleases Exo III and Exo I to degrade nucleic acids with free, unprotected 5′ and/or 3′ ends (DNA fragments with at least one unligated end and free adapters). After exonuclease treatment, the adaptor ligated templates were purified using AMPure beads according to manufacturer's instructions. The resultant adapter-ligated DNA fragments were estimated to be ˜50% asymmetrically tagged (having a hairpin adapter at one end and a linear adapter at the opposite end) and ˜50% symmetrically tagged (having the same adapter at both ends).

Target Enrichment and Sequencing:

Alzheimer's disease (AD) panel capture probes targeting DNA fragments on several chromosomes were obtained from IDT. The regions targeted by the capture probes and the number of probes specific for each region are shown in Table 4. Each of the AD capture probes were 120 bases long, biotinylated on the 5′ end, and spaced approximately ˜1 kbp apart from each other on their respective target region of interest.

TABLE 4

| Target Region | | Target | Target | Number |
|---|---|---|---|---|
| No. | Chromosome | Region Start | Region End | of probes |
| 1 | 1 | 160313062 | 160328058 | 28 |
| 2 | 1 | 227057884 | 227083244 | 29 |
| 3 | 1 | 207669472 | 207814985 | 5 |
| 4 | 2 | 127806089 | 127864903 | 69 |
| 5 | 2 | 233925035 | 233995922 | 7 |
| 6 | 4 | 90645603 | 90759484 | 10 e* |
| 7 | 5 | 88014293 | 88199922 | 55 e |
| 8 | 6 | 32485409 | 32557613 | 29 |
| 9 | 6 | 41126398 | 41130924 | 9 |
| 10 | 6 | 47445481 | 47594047 | 20 |
| 11 | 7 | 99998839 | 100027037 | 33 |
| 12 | 7 | 143087324 | 143105985 | 30 |
| 13 | 7 | 37887929 | 37939965 | 60 |
| 14 | 8 | 27168998 | 27472328 | 60 |
| 15 | 11 | 47488434 | 47544792 | 21 |
| 16 | 11 | 59939429 | 59952139 | 19 |
| 17 | 11 | 117157217 | 117186972 | 31 |
| 18 | 11 | 121322911 | 121503510 | 52 |
| 19 | 14 | 53324104 | 53417856 | 92 |
| 20 | 14 | 73603142 | 73690281 | 18e |
| 21 | 14 | 92788892 | 93154390 | 41 e |
| 22 | 17 | 42422490 | 42430148 | 12 e |
| 23 | 17 | 43971701 | 44105689 | 21 e |
| 24 | 19 | 571276 | 582869 | 17 |
| 25 | 19 | 1040088 | 1065388 | 53 |
| 26 | 19 | 45394476 | 45411909 | 24 |
| 27 | 19 | 51728312 | 51742892 | 24 |
| 28 | 20 | 54987159 | 55033515 | 47 |
| 29 | 21 | 27252972 | 27543446 | 21 e |

*“e” stands for “exon”; these probes are designed for RNA or cDNA capturing.

To separate the strands of the hairpin-ligated DNA fragments in the human genomic DNA library, a nucleic acid synthesis reaction was initiated (using a cognate synthesis primer and a wild-type phi-29 DNA polymerase) from the nucleic acid synthesis primer binding site present in the loop region of the hairpin adapter. The reaction was performed on 2.7 μg of the adapter-tagged genomic DNA library and allowed to proceed for 15 minutes. In addition to the four standard dNTPs (200 micromolar each), the reaction included dUTP (100 micromolar). The reaction was performed at 25° C. and was stopped by adding 0.05× volume of 0.1% SDS+0.02 unit/uL Proteinase K. After 5 min at RT, the sample was cleaned by AMPure purification. Primer-extended complexes were exposed to the 29 biotin-probes listed in Table 4 under nucleic acid hybridization conditions using IDT kit's hybridization buffers for 16 hours at 65° C. and then to streptavidin (SA)-beads according to the manufacturer's (IDT) protocol. After removal of the supernatant and washing, captured complexes were released and isolated from the SA-beads by incubation with Exonuclease III, Exonuclease I, and USER enzyme mix (Uracil-Specific Excision Reagent; New England Biolabs). The USER enzyme mix includes a Uracil DNA glycosylase (UDG) and an Endonuclease VIII enzyme (EndoVIII). This treatment digests the IDT probes and the nascent DNA synthesized to separate the strands. The isolated DNA fragments, released from beads, were sequenced in a Single Molecule, Real-Time (SMRT®) Sequencing reaction on a PacBio RS II instrument (Pacific Biosciences of California, Inc.) using a sequencing primer specific for the sequencing primer binding site present in the 3′ overhang region of the linear adapter.

Results:

The sequencing data returned a total of 606 reads that mapped to human genomic DNA (mapped reads). Table 5 shows the number of these mapped reads assigned to each of the AD target regions targeted by the AD capture probes. Table 5 also indicates the length of each of the mapped reads.

TABLE 5

| | Mapped Reads in | Mapped Read |
|---|---|---|
| Target Region No. | Target Region | Length |
| 1 | 0 | |
| 2 | 0 | |
| 3 | 0 | |
| 4 | 0 | |
| 5 | 0 | |
| 6 | 0 | |
| 7 | 0 | |
| 8 | 2 | 1678, 144 |
| 9 | 0 | |
| 10 | 2 | 349, 1843 |
| 11 | 4 | 52, 168, 313, 1338 |
| 12 | 1 | 1074 |
| 13 | 0 | |
| 14 | 1 | 1261 |
| 15 | 2 | 599, 130 |
| 16 | 2 | 468, 565 |
| 17 | 0 | |
| 18 | 3 | (910, 450)*, 1965, |
| | | 2231 |
| 19 | 1 | 66 |
| 20 | 0 | |
| 21 | 0 | |
| 22 | 0 | |
| 23 | 0 | |
| 24 | 0 | |
| 25 | 1 | 836 |
| 26 | 1 | 266 |
| 27 | 0 | |
| 28 | 1 | 66 |
| 29 | 0 | |

*These two reads are from the same original template, and thus are counted as one mapped read in this table.

As noted above, the total number of human mapped reads was 606. The total number of these 606 mapped reads assigned to the AD target regions is 21. Thus, 3.5% of the mapped reads are for the desired target regions. The number of mapped reads that are in excess of 200 base pairs is 16. Because of their size, these 16 on-target mapped reads cannot be derived from sequencing reads of the capture primer themselves (as opposed to captured genomic DNA templates), as they are only 120 bases long. Thus, at least 2.6% of the total mapped reads are for the desired target regions. This represents a substantial enrichment, as the percentage of on-target reads without enrichment is estimated as 0.0817% (all the target regions added together cover 2,615,794 bp and human gDNA is ˜3,200,000,000 bp).

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1. A method of enriching for nucleic acids comprising a target sequence from a mixture of nucleic acids, comprising:
providing a mixture of nucleic acids, wherein the nucleic acids comprise:
a double-stranded insert region having a first and second end, wherein one or more insert regions include a target sequence; and
a strand-linking adapter at the first end;
denaturing the double-stranded insert regions of the nucleic acids;
contacting the denatured nucleic acids to one or more capture probes comprising a capture region specific for the target sequence, wherein the contacting is under conditions that allow sequence-specific binding of the capture region to the target sequence;
isolating nucleic acids bound to the one or more capture probes;
removing the one or more capture probes from the isolated nucleic acids;
renaturing the double-stranded insert region of the isolated nucleic acids, thereby enriching for nucleic acids comprising the target sequence.

2. The method of claim 1, wherein the nucleic acids further comprise a second adapter at the second end of the double-stranded insert region.

3. The method of claim 2, wherein the second adapter is a non-strand-linking adapter.

4. (canceled)

5. The method of claim 1, wherein the capture region comprises a nucleic acid sequence complementary to one nucleic acid strand of the target region.

6. The method of claim 5, wherein the nucleic acid sequence in the capture region is an RNA sequence, wherein the removing step comprises contacting the isolated nucleic acids with an RNase that degrades RNA in an RNA/DNA heteroduplex to degrade the capture region RNA sequence.

7-8. (canceled)

9. The method of claim 5, wherein the nucleic acid sequence in the capture region is a DNA sequence, wherein the removing step comprises contacting the isolated nucleic acids with an exonuclease to degrade the capture region DNA sequence.

10-11. (canceled)

12. The method of claim 1, wherein a plurality of capture probes is contacted to the denatured nucleic acids, wherein the plurality of capture probes comprises capture regions that are specific for different target sequences.

13. (canceled)

14. The method of claim 1, wherein the strand-linking adapter is a nucleic acid hairpin adapter, wherein the hairpin adapter comprises a nucleic acid synthesis primer binding site, a sequencing primer binding site, or both.

15-16. (canceled)

17. The method of claim 3, wherein the non-strand-linking adapter is a linear nucleic acid adapter, wherein a first end of the linear nucleic acid adapter is configured to ligate to compatible double-stranded DNA ends and the second end of the linear nucleic acid adapter is protected from exonuclease digestion.

18-19. (canceled)

20. The method of claim 17, wherein the second end of the linear oligonucleotide adapter comprises a 3′ overhang region that includes a sequencing primer binding site.

21. The method of claim 17, wherein the linear nucleic acid adapter comprises a restriction enzyme cleavage site, wherein the method further comprises:
cleaving the enriched nucleic acids at the restriction enzyme cleavage site; and
ligating a second strand-linking adapter to the digested restriction enzyme cleavage site.

22. The method of claim 21, wherein the second strand-linking adapter is a second hairpin adapter, wherein the second hairpin adapter comprises a sequencing primer binding site.

23. (canceled)

24. The method of claim 15, wherein the denaturing comprises:
hybridizing a synthesis primer to the nucleic acid synthesis primer binding site in the hairpin adapter of the nucleic acids; and
placing the hybridized nucleic acids in a nucleic acid synthesis reaction mixture comprising a strand-displacing nucleic acid polymerase to generate a nascent nucleic acid strand on one strand of the double-stranded nucleic acid insert of the nucleic acids, thereby displacing the complementary strand of the nucleic acids.

25. The method of claim 24, wherein the nucleic acid synthesis reaction mixture comprises dUTP nucleotides, wherein the removing and/or renaturing steps comprises contacting the isolated nucleic acids with one or more nucleases that degrade the capture region of the capture probe and the nascent nucleic acid, wherein the one or more nucleases comprises an uracil-specific excision reagent (USER).

26. The method of claim 24, wherein the removing and/or renaturing steps comprises contacting the isolated nucleic acids with one or more nucleases that degrade the capture region of the capture probe and the nascent nucleic acid.

27-28. (canceled)

29. The method of claim 1, wherein the one or more capture probes comprise a retrieval region, wherein the retrieval region is a first member of a binding pair, wherein the isolating step comprises contacting the capture probe-contacted sample with a solid substrate comprising the binding partner of the first member of the binding pair.

30. (canceled)

31. The method of claim 29, wherein the first member of the binding pair is selected from the group consisting of: a nucleic acid sequence, biotin, avidin, streptavidin, digoxigenin, a protein, an antibody, or combinations thereof.

32-35. (canceled)

36. The method of claim 1, further comprising sequencing the enriched nucleic acids.

37. The method of claim 1, wherein the double-strand insert regions of the nucleic acids in the mixture are derived from: genomic DNA, cDNA, cell free DNA, fragmented DNA, damaged DNA, DNA form a formalin-fixed paraffin embedded (FFPE) tissue sample, DNA from a clinical sample, DNA form a tissue sample, and any combination thereof.

38. The method of claim 1, wherein the nucleic acid mixture is a multiplexed sample, and wherein the nucleic acids in the multiplexed sample comprise barcodes that allow identification of their source.

39-43. (canceled)
